(19) United States
(12) Patent Application Publication
(10) Pub. No.: US 2011/0306512 A1
(43) Pub. Date: Dec. 15, 2011

(54) Gene Expression Profiling for Identification, Monitoring, and Treatment of Osteoarthritis

(75) Inventors: Danute M. Bankaitis-Davis, Longmont, CO (US); Lisa Siconolfi, Westminster, CO (US); Kathleen Storm, Longmont, CO (US); Karl Wassmann, Dover, MA (US)

(73) Assignee: DxTerity Diagnostics, Los Angeles, CA (US)

(21) Appl. No.: 13/000,729

(22) PCT Filed: Jun. 25, 2009
(86) PCT No.: PCT/US09/48684

§ 371 Date: Aug. 31, 2011

Related U.S. Application Data
(60) Provisional application No. 61/075,539, filed on Jun. 25, 2008.

(51) Int. Cl. C40B 30/04; C12Q 1/68

(52) U.S. Cl. 506/9; 435/6.12

(57) ABSTRACT

A method is provided in various embodiments for determining a profile data set for a subject with osteoarthritis or conditions related to osteoarthritis based on a sample from the subject, wherein the sample provides a source of RNAs. The method includes using amplification for measuring the amount of RNA corresponding to at least one constituent from Tables 1-2, 4-6, and 8. The profile data set comprises the measure of each constituent, and amplification is performed under measurement conditions that are substantially repeatable.

### REFERENCE TO RELATED APPLICATIONS

This application claims the benefit of U.S. Provisional Application No.61/075,539, filed Jun. 25, 2008, the contents of which are incorporated by reference in its entirety.

### FIELD OF THE INVENTION

The present invention relates generally to the identification of biological markers associated with the identification of osteoarthritis. More specifically, the present invention relates to the use of gene expression data in the identification, monitoring and treatment of osteoarthritis and in the characterization and evaluation of conditions induced by or related to osteoarthritis erythematosus.

### BACKGROUND OF THE INVENTION

Osteoarthritis (OA), also known as degenerative arthritis or degenerative joint disease, is a condition in which low-grade inflammation results in pain in the joints, caused by wearing of the cartilage that covers and acts as a cushion inside joints. As the bone surfaces become less well protected by cartilage, the patient experiences pain upon weight bearing, including walking and standing. Due to decreased movement because of the pain, regional muscles may atrophy, and ligaments may become more lax.

OA is the commonest form of joint disease and a leading cause of disability in the elderly. It is strongly associated with increasing age and it is estimated that 80% of the population will have radiographic evidence of OA by age 65, although only 60% of those will be symptomatic. Even though the radiographic changes of OA are often asymptomatic, symptomatic knee OA, with an estimated incidence of 240/100,000 person years, is the most frequent cause of dependency in lower limb tasks, especially in the elderly. It causes 68 million work loss days per year and more than 5% of the annual retirement rate. It has considerable economic and societal costs, in terms of work loss, and hospital admission. Furthermore, OA is the most frequent reason for joint replacement at a cost to the community of billions of dollars per year.

Information on any condition of a particular patient and a patient's response to types and dosages of therapeutic or nutritional agents has become an important issue in clinical medicine today not only from the aspect of efficiency of medical practice for the health care industry but for improved outcomes and benefits for the patients. The advent of genomics offers the potential for elucidation of underlying mechanisms of OA progression and the applying this knowledge to the clinical and drug-development settings. Altered gene-expression patterns precede synthesis and release of cytokine proteins and other enzymatically important signals. Therefore, the analysis of specific mRNA species associated with these changes may provide the earliest indication of disease progression.

Growth in genomics has exploded in recent years with its promise of improving drug discovery and patient care. Multiple methods are now available for detecting and quantifying gene-expression levels, including northern blots, S1 nuclease protection, differential display, cDNA library sequencing, quantitative PCR, and array-based techniques (cDNA and oligonucleotide arrays). Most commercially driven genomics programs focus on microarray technology for assessment of differential gene-expression patterns. Although the capability of examining mRNA from a large number of genes simultaneously makes this technique appear attractive, the expression levels of the vast majority of those genes remain unchanged, while the amount of data generated is daunting (Kothapalli et al., 2002; Simon et al., 2003). Currently, researchers are attempting to circumvent this problem by developing complex statistical methods of dealing with overwhelming quantities of data (Allison et al., 2006; Zhao et al., 2001; Fellenberg et al., 2001). Nevertheless, the signal-to-noise problem for microarrays that monitor 10,000 genes at one time remains a significant barrier. A false-response rate of only 1% in this case will result in 100 false measurements (Mills and Gordon, 2001). Additional problems arise from the low specificity of microarray probes and lack of probe specificity for different isoforms of a gene (Kothapalli et al., 2002).

Currently, no effective disease-modifying medical remedies for OA exist. Typical treatment consists of medication or other interventions that can reduce the pain of OA and thereby improve the function of the joint, such as NSAIDs, local injections of glucocorticoid or hyaluronan, and in severe cases, joint replacement surgery. Disease-modifying medical interventions have been developed for other age-related disorders such as osteoporosis, but progress in the osteoarthritis field has been obfuscated by absence of biomarkers for disease activity. While a variety of biochemical assays of cartilage and bone derived breakdown products have been developed and tested, none have exhibited sufficient predictivity to inform clinical decision-making or facilitate drug development. Thus, a testing capability that can discriminate OA patients from healthy individuals, measure disease activity and identify patients exhibiting progression is needed to facilitate the development of disease-modifying interventions to for osteoarthritis. The present invention meets these needs and other needs.

### SUMMARY OF THE INVENTION

The invention is in based in part upon the identification of gene expression profiles (Precision Profiles™) associated with osteoarthritis. These genes are referred to herein as osteoarthritis associated genes. More specifically, the invention is based upon the surprising discovery that detection of as few as two osteoarthritis associated genes is capable of identifying individuals with or without osteoarthritis with at least 75% accuracy. More particularly, the invention is based upon the surprising discovery that the methods provided by the invention are capable of detecting osteoarthritis by assaying blood samples.

In various aspects the invention provides methods of evaluating the presence or absence (e.g., diagnosing or prognosing) of osteoarthritis, based on a sample from the subject, the sample providing a source of RNAs, and determining a quantitative measure of the amount of at least one constituent of any constituent of Tables 1-2, 4-6, or 8, and arriving at a measure of each constituent.

Also provided are methods of assessing or monitoring the response to therapy in a subject having osteoarthritis, based on a sample from the subject, the sample providing a source of RNAs, determining a quantitative measure of the amount of at least one constituent of any constituent of Tables 1-2, 4-6, or 8, and arriving at a measure of each constituent.

In a further aspect the invention provides methods of monitoring the progression of osteoarthritis in a subject, based on a sample from the subject, the sample providing a source of RNAs, by determining a quantitative measure of the amount of at least one constituent of any constituent of Tables Tables 1-2, 4-6, or 8 as a distinct RNA constituent in a sample obtained at a first period of time to produce a first subject data set and determining a quantitative measure of the amount of at least one constituent of any constituent of Tables 1-2, 4-6, or 8 as a distinct RNA constituent in a sample obtained at a second period of time to produce a second subject data set. Optionally, the constituents measured in the first sample are the same constituents measured in the second sample. The first subject data set and the second subject data set are compared allowing the progression of osteoarthritis in a subject to be determined. The second subject is taken e.g., one day, one week, one month, two months, three months, 1 year, 2 years, or more after the first subject sample. Optionally the first subject sample is taken prior to the subject receiving treatment, e.g. non-steroidal anti-inflammatory drugs (NSAIDs, e.g., diclofenac, ibuprofen, and naproxen), COX-2 selective inhibitors (e.g., celecoxib, rofecoxib, and valdecoxib), acetaminophen, local injections of glucocorticoid or hyaluronan, and/or lidocaine, and the second subject sample is taken after treatment.

In various aspects the invention provides a method for determining a profile data set for characterizing a subject with osteoarthritis or conditions related to osteoarthritis based on a sample from the subject, the sample providing a source of RNAs, by using amplification for measuring the amount of RNA in a panel of constituents including at least 2 constituents from any of Tables 1-2, 4-6, and 8, and arriving at a measure of each constituent. The profile data set contains the measure of each constituent of the panel.

The methods of the invention further include comparing the quantitative measure of the constituent in the subject derived sample to a reference value or a baseline value, e.g. baseline data set. The reference value is for example an index value. Comparison of the subject measurements to a reference value allows for the present or absence of osteoarthritis to be determined, response to therapy to be monitored or the progression of osteoarthritis to be determined. For example, a similarity in the subject data set compares to a baseline data set derived from a subject having osteoarthritis indicates that presence of osteoarthritis or response to therapy that is not efficacious. Whereas a similarity in the subject data set compares to a baseline data set derived from a subject not having osteoarthritis indicates the absence of osteoarthritis or response to therapy that is efficacious. In various embodiments, the baseline data set is derived from one or more other samples from the same subject, taken when the subject is in a biological condition different from that in which the subject was at the time the first sample was taken, with respect to at least one of age, nutritional history, medical condition, clinical indicator, medication, physical activity, body mass, and environmental exposure, and the baseline profile data set may be derived from one or more other samples from one or more different subjects.

The baseline data set or reference values may be derived from one or more other samples from the same subject taken under circumstances different from those of the first sample, and the circumstances may be selected from the group consisting of (i) the time at which the first sample is taken (e.g., before, after, or during treatment osteoarthritis treatment), (ii) the site from which the first sample is taken, (iii) the biological condition of the subject when the first sample is taken.

The measure of the constituent is increased or decreased in the subject compared to the expression of the constituent in the reference, e.g., normal reference sample or baseline value. The measure is increased or decreased 10%, 25%, 50% compared to the reference level. Alternately, the measure is increased or decreased 1, 2, 5 or more fold compared to the reference level.

In various aspects of the invention the methods are carried out wherein the measurement conditions are substantially repeatable, particularly within a degree of repeatability of better than ten percent, five percent or more particularly within a degree of repeatability of better than three percent, and/or wherein efficiencies of amplification for all constituents are substantially similar, more particularly wherein the efficiency of amplification is within ten percent, more particularly wherein the efficiency of amplification for all constituents is within five percent, and still more particularly wherein the efficiency of amplification for all constituents is within three percent or less.

In addition, the one or more different subjects may have in common with the subject at least one of age group, gender, ethnicity, geographic location, nutritional history, medical condition, clinical indicator, medication, physical activity, body mass, and environmental exposure. A clinical indicator may be used to assess osteoarthritis or a condition related to osteoarthritis of the one or more different subjects, and may also include interpreting the calibrated profile data set in the context of at least one other clinical indicator, wherein the at least one other clinical indicator includes blood chemistry, X-ray or other radiological or metabolic imaging technique, molecular markers in the blood, other chemical assays, and physical findings.

At least 30, 20, 15, 12, 10, 8, 6, 5, 4, 3, 2 or fewer constituents are measured. Preferably, at least one constituent is measured. For example, the constituent is from any of Tables 1-2, 4-6, and 8 and is selected from the group consisting of IL6R, TNFAIP3, EGR1, TGFB1, IL4R, PF4, TGFBR2, IL1RN, IL1B, IL18BP, IL13RA1, MMP9, TNFRSF1A, IL1R1, IL18R1, IFNGR1, TGFBR1, TNFAIP6, TGFB3, and IL10. In a particular embodiment, at least 2 constituents from any of Tables 1-2, 4-6, and 8 are measured. For example, 1) IL6R and PF4, or 2) EGR1 and TNFAIP3 are measured.

The constituents are selected so as to distinguish from a normal reference subject and a osteoarthritis-diagnosed subject. Alternatively, the panel of constituents is selected as to permit characterizing the severity of osteoarthritis in relation to a normal subject over time so as to track osteoarthritis recurrence. Thus in some embodiments, the methods of the invention are used to determine efficacy of treatment of a particular subject.

Preferably, the panel of constituents are selected so as to distinguish, e.g., classify between a normal and an osteoarthritis-diagnosed subject with at least 75%, 80%, 85%, 90%, 95%, 97%, 98%, 99% or greater accuracy. By “accuracy” is meant that the method has the ability to distinguish, e.g., classify, between subjects having osteoarthritis or conditions associated with osteoarthritis, and those that do not. Accuracy is determined for example by comparing the results of the Gene Precision Profiling™ to standard accepted clinical methods of diagnosing osteoarthritis, e.g., physical examination of joint appearance and joint symptoms, x-ray, magnetic resonance imaging (MRI), arthrocentesis, and arthroscopy.

Additionally, the invention includes a biomarker for predicting individual response to osteoarthritis treatment (wherein osteoarthritis treatment includes photoprovocation and an agent for the treatment of osteoarthritis) in a subject having osteoarthritis or a condition related to osteoarthritis comprising at least one constituent of any constituent of Tables 1-2, 4-6, and 8. Optimally, the biomarker comprises IL6R, TNFAIP3, EGR1, TGFB1, IL4R, PF4, TGFBR2, IL1RN, IL1B, IL18BP, IL13RA1, MMP9, TNFRSF1A, IL1R1, IL18R1, TNF, IFNGR1, TGFBR1, TNFAIP6, TGFB3, and IL10.

By osteoarthritis or conditions related to osteoarthritis is meant any low-grade inflammation resulting in pain in the joints caused by wearing of the cartilage that covers and acts as a cushion inside joints, including primary osteoarthritis and secondary osteoarthritis caused by congential disorders (e.g., congential hip luxation and abnormally formed joints), cracking joints, diabetes, inflammatory diseases (e.g., Perthe's Disease, Lyme Disease), chronic forms of arthritis (e.g., gout, costochondritis, and rheumatoid arthritis), injury to joints, hormonal disorders, ligamentous deterioration, obesity, osteoporosis, and surgery to joint structures.

The sample is any sample derived from a subject which contains RNA. For example, the sample is blood, a blood fraction, body fluid, a population of cells (e.g., bone cells) or tissue (e.g., osteoarthritic tissue) from the subject, or circulating endothelial cells found in the blood.

Optionally one or more other samples can be taken over an interval of time that is at least one month between the first sample and the one or more other samples, or taken over an interval of time that is at least twelve months between the first sample and the one or more samples, or they may be taken pre-therapy intervention or post-therapy intervention. In such embodiments, the first sample may be derived from blood and the baseline profile data set may be derived from tissue or body fluid of the subject other than blood. Alternatively, the first sample is derived from tissue or bodily fluid of the subject and the baseline profile data set is derived from blood.

Also included in the invention are kits for the detection of osteoarthritis in a subject, containing at least one reagent for the detection or quantification of any constituent measured according to the methods of the invention and instructions for using the kit.

All of the forgoing embodiments are carried out wherein the measurement conditions are substantially repeatable, particularly within a degree of repeatability of better than ten percent, five percent or more particularly within a degree of repeatability of better than three percent, and/or wherein efficiencies of amplification for all constituents are substantially similar, more particularly wherein the efficiency of amplification is within ten percent or less, more particularly wherein the efficiency of amplification for all constituents is within five percent or less, and still more particularly wherein the efficiency of amplification for all constituents is within three percent or less.

Additionally the invention includes storing the profile data set in a digital storage medium. Optionally, storing the profile data set includes storing it as a record in a database.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a graphical representation of the 2-gene model IL6R and PF4, based on the Precision Profile™ for Osteoarthritis (Table 1), capable of distinguishing between subjects afflicted with osteoarthritis and normal subjects. IL6R values are plotted along the Y-axis. PF4 values are plotted along the X-axis.

FIG. 2 is a graphical representation of the 2-gene model EGR1 and TNFAIP3, based on the Precision Profile™ for Osteoarthritis (Table 1), capable of distinguishing between subjects afflicted with osteoarthritis and normal subjects. EGR1 values are plotted along the Y-axis. TNFAIP3 values are plotted along the X-axis.

### DETAILED DESCRIPTION OF SPECIFIC EMBODIMENTS

Definitions

The following terms shall have the meanings indicated unless the context therwise requires:

“Algorithm” is a set of rules for describing a biological condition. The rule set may be defined exclusively algebraically but may also include alternative or multiple decision points requiring domain-specific knowledge, expert interpretation or other clinical indicators.

An “agent” is a “composition” or a “stimulus”, as those terms are defined herein, or a combination of a composition and a stimulus.

“Amplification” in the context of a quantitative RT-PCR assay is a function of the number of DNA replications that are tracked to provide a quantitative determination of its concentration. “Amplification” here refers to a degree of sensitivity and specificity of a quantitative assay technique. Accordingly, amplification provides a measurement of concentrations of constituents that is evaluated under conditions wherein the efficiency of amplification and therefore the degree of sensitivity and reproducibility for measuring all constituents is substantially similar (i.e., within ten percent or less, preferably within five percent or less, even more preferably within three percent or less).

A “baseline profile data set” is a set of values associated with constituents of a Gene Expression Panel resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

A “biological condition” of a subject is the condition of the subject in a pertinent realm that is under observation, and such realm may include any aspect of the subject capable of being monitored for change in condition, such as health; disease including osteoarthritis; cancer; trauma; aging; infection; tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

“Body fluid” of a subject includes blood, urine, spinal fluid, lymph, mucosal secretions, prostatic fluid, semen, haemolymph or any other body fluid known in the art for a subject.

“Calibrated profile data set” is a function of a member of a first profile data set and a corresponding member of a baseline profile data set for a given constituent in a panel.

A “clinical indicator” is any physiological datum used alone or in conjunction with other data in evaluating the physiological condition of a collection of cells or of an organism. This term includes pre-clinical indicators.

A “composition” includes a chemical compound, a nutriceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.

To “derive” a profile data set from a sample includes determining a set of values associated with constituents of a Gene Expression Panel either (i) by direct measurement of such constituents in a biological sample or (ii) by measurement of such constituents in a second biological sample that has been exposed to the original sample or to matter derived from the original sample.

“Distinct RNA or protein constituent” in a panel of constituents is a distinct expressed product of a gene, whether RNA or protein. An “expression” product of a gene includes the gene product whether RNA or protein resulting from translation of the messenger RNA.

A “Gene Expression Panel” (Precision Profile™) is an experimentally verified set of constituents, each constituent being a distinct expressed product of a gene, whether RNA or protein, wherein constituents of the set are selected so that their measurement provides a measurement of a targeted biological condition.

A “Gene Expression Profile” is a set of values associated with constituents of a Gene Expression Panel resulting from evaluation of a biological sample (or population or set of samples).

A “Gene Expression Profile Inflammatory Index” is the value of an index function that provides a mapping from an instance of a Gene Expression Profile into a single-valued measure of inflammatory condition.

A Gene Expression Profile Osteoarthritis Index” is the value of an index function that provides a mapping from an instance of a Gene Expression Profile into a single-valued measure of an osteoarthritis condition.

The “health” of a subject includes mental, emotional, physical, spiritual, allopathic, naturopathic and homeopathic condition of the subject.

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

“Inflammation” is used herein in the general medical sense of the word and may be an acute or chronic; simple or suppurative; localized or disseminated; cellular and tissue response initiated or sustained by any number of chemical, physical or biological agents or combination of agents.

“Inflammatory state” is used to indicate the relative biological condition of a subject resulting from inflammation, or characterizing the degree of inflammation.

A “large number” of data sets based on a common panel of genes is a number of data sets sufficiently large to permit a statistically significant conclusion to be drawn with respect to an instance of a data set based on the same panel.

The term “osteoarthritis treatment”encompasses both a composition or other agent for the amelioration of the disease and/or symptoms of osteoarthritis, and stimulus for the induction of the disease and/or symptoms of osteoarthritis.

A “normal” subject is a subject who has not been diagnosed with osteoarthritis, or one who is not suffering from osteoarthritis.

A “normative” condition of a subject to whom a composition is to be administered means the condition of a subject before administration, even if the subject happens to be suffering from a disease.

The term “osteoarthritis” is a condition in which low-grade inflammation results in pain in the joints caused by wearing of the cartilage that covers and acts as a cushion inside joints, and is used to indicate degenerative arthritis or degenerative joint disease. The term osteoarthritis includes primary osteoarthritis and secondary osteoarthritis caused by congential disorders (e.g., congential hip luxation and abnormally formed joints), cracking joints, diabetes, inflammatory diseases (e.g., Perthe's Disease, Lyme Disease), chronic forms of arthritis (e.g., gout, costochondritis, and rheumatoid arthritis), injury to joints, hormonal disorders, ligamentous deterioration, obesity, osteoporosis, and surgery to joint structures.

A “panel” of genes is a set of genes including at least two constituents.

A “population of cells” refers to any group of cells wherein there is an underlying commonality or relationship between the members in the population of cells, including a group of cells taken from an organism or from a culture of cells or from a biopsy, for example.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art.

A “set” or “population” of samples or subjects refers to a defined or selected group of samples or subjects wherein there is an underlying commonality or relationship between the members included in the set or population of samples or subjects.

A “Signature Profile” is an experimentally verified subset of a Gene Expression Profile selected to discriminate a biological condition, agent or physiological mechanism of action.

A “Signature Panel” is a subset of a Gene Expression Panel (Precision Profile™), the constituents of which are selected to permit discrimination of a biological condition, agent or physiological mechanism of action.

A “subject” is a cell, tissue, or organism, human or non-human, whether in vivo, ex vivo or in vitro, under observation. As used herein, reference to evaluating the biological condition of a subject based on a sample from the subject, includes using blood or other tissue sample from a human subject to evaluate the human subject's condition; it also includes, for example, using a blood sample itself as the subject to evaluate, for example, the effect of therapy or an agent upon the sample.

A “stimulus” includes (i) a monitored physical interaction with a subject, for example use of an agent to induce a disease or disease symptom, e.g., ultraviolet A or B to induce a skin reaction (photoprovocation), or treatment of disease or disease symptom with an agent; and (ii) any monitored physical, mental, emotional, or spiritual activity or inactivity of a subject.

“Therapy” includes all interventions whether biological, chemical, physical, metaphysical, or combination of the foregoing, intended to sustain or alter the monitored biological condition of a subject.

The PCT patent application publication number WO 01/25473, published Apr. 12, 2001, entitled “Systems and Methods for Characterizing a Biological Condition or Agent Using Calibrated Gene Expression Profiles,” filed for an invention by inventors herein, and which is herein incorporated by reference, discloses the use of Gene Expression Panels for the evaluation of (i) biological condition (including with respect to health and disease) and (ii) the effect of one or more agents on biological condition (including with respect to health, toxicity, therapeutic treatment and drug interaction).

In particular, Gene Expression Panels (Precision Profiles™) may be used for measurement of therapeutic efficacy of natural or synthetic compositions or stimuli that may be formulated individually or in combinations or mixtures for a range of targeted biological conditions; prediction of toxicological effects and dose effectiveness of a composition or mixture of compositions for an individual or for a population or set of individuals or for a population of cells; determination of how two or more different agents administered in a single treatment might interact so as to detect any of synergistic, additive, negative, neutral or toxic activity; performing pre-clinical and clinical trials by providing new criteria for pre-selecting subjects according to informative profile data sets for revealing disease status; and conducting preliminary dosage studies for these patients prior to conducting phase 1 or 2 trials. These Gene Expression Panels (Precision Profiles™) may be employed with respect to samples derived from subjects in order to evaluate their biological condition.

The present invention provides Gene Expression Panels (Precision Profiles™) for the evaluation or characterization of osteoarthritis and conditions related to osteoarthritis in a subject. In addition, the Gene Expression Profiles described herein also provided the evaluation of the effect of one or more agents for the treatment of osteoarthritis and conditions related to osteoarthritis.

The Gene Expression Panels (Precision Profiles™) are refered to herein as the “Precision Profile™ for Osteoarthritis” and the “Precision Profile™ for Inflammatory Response”. A Precision Profile™ for Osteoarthritis includes one or more genes, e.g., constituents, listed in Tables 1-2, 4-6, and 8. A Precision Profile™ for Inflammatory Response includes one or more genes, e.g., constituents, listed in Table 2. Each gene of the Precision Profile™ for Osteoarthritis and Precision Profile™ for Inflammatory Response is refered to herein as an osteoarthritis associated gene or an osteoarthritis associated constituent.

The evaluation or characterization of osteoarthritis is defined to be diagnosing osteoarthritis, assessing the risk of developing osteoarthritis or assessing the prognosis of a subject with osteoarthritis. Similarly, the evaluation or characterization of an agent for treatment of osteoarthritis includes identifying agents suitable for the treatment of osteoarthritis. The agents can be compounds known to treat osteoarthritis or compounds that have not been shown to treat osteoarthritis.

Osteoarthritis and conditions related to osteoarthritis is evaluated by determinining the level of expression (e.g., a quantitative measure) of one or more osteoarthritis genes. The level of expression is determined by any means known in the art, such as for example quantitative PCR. The measurement is obtained under conditions that are substantially repeatable. Optionally, the qualitative measure of the constituent is compared to a baseline level (e.g. baseline profile set). A baseline level is a level of expression of the constituent in one or more subjects known not to be suffering from osteoarthritis (e.g., normal, healthy individual(s)). Alternatively, the baseline level is derived from one or more subjects known to be suffering from osteoarthritis. Optionally, the baseline level is derived from the same subject from which the first measure is derived. For example, the baseline is taken from a subject prior to receiving treatment for osteoarthritis, or at different time periods during a course of treatment. Such methods allow for the evalution of a particular treatment for a selected individual. Comparison can be performed on test (e.g., patient) and reference samples (e.g., baseline) measured concurrently or at temporally distinct times. An example of the latter is the use of compiled expression information, e.g., a gene expression database, which assembles information about expression levels of osteoarthritis genes.

A change in the expression pattern in the patient-derived sample of an osteoarthritis gene compared to the normal baseline level indicates that the subject is suffering from or is at risk of developing osteoarthritis. In contrast, when the methods are applied prophylactically, a similar level compared to the normal control level in the patient-derived sample of an osteoarthritis gene indicates that the subject is not suffering from or at risk of developing osteoarthritis. Whereas, a similarity in the expression pattern in the patient-derived sample of an osteoarthritis gene compared to the osteoarthritis baseline level indicates that the subject is suffering from or is at risk of developing osteoarthritis.

Expression of an effective amount of an osteoarthritis gene also allows for the course of treatment of osteoarthritis to be monitored. In this method, a biological sample is provided from a subject undergoing treatment, e.g., if desired, biological samples are obtained from the subject at various time points before, during, or after treatment. Expression of an effective amount of an osteoarthritis gene is then determined and compared to baseline profile. The baseline profile may be taken or derived from one or more individuals who have been exposed to the treatment. Alternatively, the baseline level may be taken or dervived from one or more individuals who have not been exposed to the treatment. For example, samples may be collected from subjects who have received initial treatment for osteoarthritis and subsequent treatment for osteoarthritis to monitor the progress of the treatment.

Differences in the genetic makeup of individuals can result in differences in their relative abilities to metabolize various drugs. Accordingly, the Precision Profile™ for Osteoarthritis (Table 1) and the Precision Profile™ for Inflammatory Response (Table 2) disclosed herein allow for a putative therapeutic or prophylactic to be tested from a selected subject in order to determine if the agent is a suitable for treating or preventing osteoarthritis in the subject. Additionally, other genes known to be associated with toxicity may be used. By suitable for treatment is meant determining whether the agent will be efficacious, not efficacious, or toxic for a particular individual. By toxic it is meant that the manifestations of one or more adverse effects of a drug when administered therapeutically. For example, a drug is toxic when it disrupts one or more normal physiological pathways.

To identify a therapeutic that is appropriate for a specific subject, a test sample from the subject is exposed to a candidate therapeutic agent, and the expression of one or more of osteoarthritis genes is determined. A subject sample is incubated in the presence of a candidate agent and the pattern of osteoarthritis gene expression in the test sample is measured and compared to a baseline profile, e.g., an osteoarthritis baseline profile or a non-osteoarthritis baseline profile or an index value. The test agent can be any compound or composition. For example, the test agent is a compound known to be useful in the treatment of osteoarthritis. Alternatively, the test agent is a compound that has not previously been used to treat osteoarthritis.

If the reference sample, e.g., baseline is from a subject that does not have osteoarthritis a similarity in the pattern of expression of osteoarthritis genes in the test sample compared to the reference sample indicates that the treatment is efficacious. Whereas a change in the pattern of expression of osteoarthritis genes in the test sample compared to the reference sample indicates a less favorable clinical outcome or prognosis.

By “efficacious” is meant that the treatment leads to a decrease of a sign or symtptom of osteoarthritis in the subject or a change in the pattern of expression of an osteoarthritis gene such that the gene expression pattern has an increase in similarity to that of a normal baseline pattern. Assessment of osteoarthritis is made using standard clinical protocols. Efficacy is determined in association with any known method for diagnosing or treating osteoarthritis.

Agents that are toxic for a specific subject are identified by exposing a test sample from the subject to a candidate agent, and the expression of one or more osteoarthritis genes is determined. A subject sample is incubated in the presence of a candidate agent and the pattern of osteoarthritis gene expression in the test sample is measured and compared to a baseline profile, e.g., an osteoarthritis baseline profile or a non-osteoarthritis baseline profile or an index value. The test agent can be any compound or composition. For example, the test agent is a compound known to be useful in the treatment of osteoarthritis. Alternatively, the test agent is a compound that has not previously been used to treat osteoarthritis.

If the reference sample, e.g., baseline is from a subject in whom the candidate agent is not toxic a similarity in the pattern of expression of osteoarthritis genes in the test sample compared to the reference sample indicates that the candidate agent is not toxic for the particular subject. Whereas a change in the pattern of expression of osteoarthritis genes in the test sample compared to the reference sample indicates that the candidate agent is toxic.

A Gene Expression Panel (Precision Profile™) is selected in a manner so that quantitative measurement of RNA or protein constituents in the Panel constitutes a measurement of a biological condition of a subject. In one kind of arrangement, a calibrated profile data set is employed. Each member of the calibrated profile data set is a function of (i) a measure of a distinct constituent of a Gene Expression Panel (Precision Profile™) and (ii) a baseline quantity.

It has been discovered that valuable and unexpected results may be achieved when the quantitative measurement of constituents is performed under repeatable conditions (within a degree of repeatability of measurement of better than twenty percent, and preferably ten percent or better, more preferably five percent or better, and more preferably three percent or better). For the purposes of this description and the following claims, a degree of repeatability of measurement of better than twenty percent as providing measurement conditions that are “substantially repeatable”. In particular, it is desirable that each time a measurement is obtained corresponding to the level of expression of a constituent in a particular sample, substantially the same measurement should result for substantially the same level of expression. In this manner, expression levels for a constituent in a Gene Expression Panel (Precision Profile™) may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar as defined herein. When both of these criteria are satisfied, then measurement of the expression level of one constituent may be meaningfully compared with measurement of the expression level of another constituent in a given sample and from sample to sample.

Additional embodiments relate to the use of an index or algorithm resulting from quantitative measurement of constituents, and optionally in addition, derived from either expert analysis or computational biology (a) in the analysis of complex data sets; (b) to control or normalize the influence of uninformative or otherwise minor variances in gene expression values between samples or subjects; (c) to simplify the characterization of a complex data set for comparison to other complex data sets, databases or indices or algorithms derived from complex data sets; (d) to monitor a biological condition of a subject; (e) for measurement of therapeutic efficacy of natural or synthetic compositions or stimuli that may be formulated individually or in combinations or mixtures for a range of targeted biological conditions; (f) for predictions of toxicological effects and dose effectiveness of a composition or mixture of compositions for an individual or for a population or set of individuals or for a population of cells; (g) for determination of how two or more different agents administered in a single treatment might interact so as to detect any of synergistic, additive, negative, neutral of toxic activity (h) for performing pre-clinical and clinical trials by providing new criteria for pre-selecting subjects according to informative profile data sets for revealing disease status and conducting preliminary dosage studies for these patients prior to conducting phase 1 or 2 trials.

Gene expression profiling and the use of index characterization for a particular condition or agent or both may be used to reduce the cost of phase 3 clinical trials and may be used beyond phase 3 trials; labeling for approved drugs; selection of suitable medication in a class of medications for a particular patient that is directed to their unique physiology; diagnosing or determining a prognosis of a medical condition or an infection which may precede onset of symptoms or alternatively diagnosing adverse side effects associated with administration of a therapeutic agent; managing the health care of a patient; and quality control for different batches of an agent or a mixture of agents.

### The Subject

The methods disclosed here may be applied to cells of humans, mammals or other organisms without the need for undue experimentation by one of ordinary skill in the art because all cells transcribe RNA and it is known in the art how to extract RNA from all types of cells.

A subject can include those exhibiting symptoms of OA, including but not limited to chronic pain, causing loss of mobility and often stiffness (wherein pain is generally described as a sharp ache, or a burning sensation in the associated muscles and tendons, “crepitus (a crackling noise when the affected joint is moved or touched), muscle spasm and contractions in the tendons, and fluid filled joints.

A subject can also include those who have not been previously diagnosed as having osteoarthritis or a condition related to osteoarthritis. Alternatively, a subject can also include those who have already been diagnosed as having osteoarthritis or a condition related to osteoarthritis. While there are no methods available to detect OA in its early and potentially treatable stages, diagnosis of osteoarthritis may be made, for example, from any one or combination of the following procedures: physical examination of joint appearance and joint symptoms, x-ray, magnetic resonance imaging (MRI), arthrocentesis, and arthroscopy. Optionally, the subject has previously been treated with a therapeutic agent to manage pain and/or inflammation aassociated with osteoarthritis, including but not limited to therapeutic agents for the treatment of osteoarthritis, such as high dosages of non-steroidal anti-inflammatory drugs (NSAIDs, e.g., diclofenac, ibuprofen, and naproxen), COX-2 selective inhibitors (e.g., celecoxib, rofecoxib, and valdecoxib), acetaminophen, local injections of glucocorticoid or hyaluronan, and/or lidocaine.

A subject can also include those who are suffering from, or at risk of developing osteoarthritis or a condition related to osteoarthritis, such as those who exhibit known risk factors for the development or progression osteoarthritis. For example, known risk factors for osteoarthritis include but are not limited to: older age, higher body mass index (BMI), higher bone mineral density (BMD), altered subchondral bone turnover, sub-optimal levels of Vitamin-D intake, altered Vitamin-D receptor genotype, inflammatory synovitis. Risk factors associated with the progeression of OA may vary depending on which joint is involved. For example, high BMI and varus or valgus knee deformity is associated with the progression of knee OA; night pain, the presence of femoral osteophytes, and subchondral sclerosis in females is associated with hip OA; and older age is associated with the progression of hand OA.

### Selecting Constituents of a Gene Expression Panel (Precision Profile™)

The general approach to selecting constituents of a Gene Expression Panel (Precision Profile™) has been described in PCT application publication number WO 01/25473, incorporated herein in its entirety. A wide range of Gene Expression Panels (Precision Profiles™) have been designed and experimentally validated, each panel providing a quantitative measure of biological condition that is derived from a sample of blood or other tissue. For each panel, experiments have verified that a Gene Expression Profile using the panel's constituents is informative of a biological condition. (It has also been demonstrated that in being informative of biological condition, the Gene Expression Profile is used, among other things, to measure the effectiveness of therapy, as well as to provide a target for therapeutic intervention).

Tables 1-2, 4-6, and 8 listed below, include relevant genes which may be selected for a given Precision Profile™, such as the Precision Profiles™ demonstrated herein to be useful in the evaluation of osteoarthritis and conditions related to osteoarthritis. Table 1 is a panel of genes whose expression is associated with osteoarthritis or conditions related to osteoarthritis. The genes listed in Table 1 were selected through a synthesis of the literature on other OA gene expression studies in tissue and blood and by review of Source MDx in-house datasets on OA and healthy patients. There have been several studies investigating gene expression levels in cartilage, bone and synovium of OA and healthy subjects. These studies have identified several genes that are related to OA onset and progression and warrant further investigation. In addition, one study has been able to show blood-based biomarkers in mild OA (Marshall et al, 2005). A thorough review of these studies will assist in additional gene panel selection for osteoarthritis gene expression studies.

Table 2 is a panel of genes whose expression is associated with inflammatory response. The disease osteoarthritis involves inflammation that can affect any joint in the human body. Although systemic inflammation is not a defining characteristic of OA, changes in the systemic inflammatory system in response to OA development and progression are highly probable and can be measured by a highly sensitive assay. As such, both the osteoarthritis genes listed in Table 1 and the inflammatory response genes listed in Table 2 can be used to detect osteoarthritis and distinguish between subjects suffering from osteoarthritis and Source MDx normal subjects.

In general, panels may be constructed and experimentally validated by one of ordinary skill in the art in accordance with the principles articulated in the present application.

### Design of Assays

Real-time PCR offers a number of advantages for the diagnostic development process compared with current gene expression analysis technologies. Microarrays are less sensitive than PCR and are even slightly less sensitive than is northern blotting (Taniguchi et al., 2001). Minor changes in gene expression may have serious clinical relevance and that the increased sensitivity of PCR affords a distinct advantage for its use. In addition, the signals generated from a microarray are contingent upon the amount of sample on the capture layer. Therefore, the signal is most often read as either on or off, with a narrow range of linearity. Quantitative PCR, on the other hand, has an extremely wide dynamic range. This allows the researcher to simultaneously study a number of genes with widely divergent expression levels.

Wide expression ranges among genes require an analytical method with great dynamic range. The PCR cycle number at which a fluorescent signal is first reliably detected by the Applied Biosystems Prism 7900 Sequence Detection System (Foster City, Calif.) is defined as the cycle threshold or C_{T}. The C_{T} is dependent upon the amount of specific input cDNA amplified in the reaction. Amplification of cDNA present at low levels requires more PCR cycles to generate a detectable signal than does amplification of cDNA present at relatively higher levels. Because it takes more PCR cycles to detect a low abundant cDNA than to detect a high abundant cDNA, C_{T} values are inversely proportional to gene-expression levels (Siebert, 1999; Livak and Schmittgen, 2001). The difference between the C_{T} for the test cDNA and the calibration standard cDNA is presented as a delta C_{T} (ΔC_{T}) value. The relative mRNA concentration increases with lower ΔCT values, 2-fold per ΔC_{T}, so that a ΔC_{T} of 15 represents 210 more mRNA than a ΔCT of 25.

The gene expression analysis methods of the present invention are consistent within runs and over time. These methods for measuring gene expression are significantly more precise, reproducible, and consistent across panels of genes than previously known or thought possible. The assays prescribed by the methods of the present invention enable the measurement of gene-expression responses with high precision, which is necessary to give data clinical utility. These assays are backed by a growing molecular medicine knowledge system that includes comparative datasets on normal subjects, specific diseases and responses to commonly prescribed therapies.

Typically, a sample is run through a panel in replicates of three for each target gene (assay); that is, a sample is divided into aliquots and for each aliquot the concentrations of each constituent in a Gene Expression Panel (Precision Profile™) is measured. From over a total of 900 constituent assays, with each assay conducted in triplicate, an average coefficient of variation was found (standard deviation/average)*100, of less than 2 percent among the normalized ΔC_{T} measurements for each assay (where normalized quantitation of the target mRNA is determined by the difference in threshold cycles between the internal control (e.g., an endogenous marker such as 18S rRNA, or an exogenous marker) and the gene of interest. This is a measure called “intra-assay variability”. Assays have also been conducted on different occasions using the same sample material. This is a measure of “inter-assay variability”. Preferably, the average coefficient of variation of intra- assay variability or inter-assay variability is less than 20%, more preferably less than 10%, more preferably less than 5%, more preferably less than 4%, more preferably less than 3%, more preferably less than 2%, and even more preferably less than 1%.

It has been determined that it is valuable to use the quadruplicate or triplicate test results to identify and eliminate data points that are statistical “outliers”; such data points are those that differ by a percentage greater, for example, than 3% of the average of all three or four values. Moreover, if more than one data point in a set of three or four is excluded by this procedure, then all data for the relevant constituent is discarded.

### Measurement of Gene Expression for a Constituent in the Panel

For measuring the amount of a particular RNA in a sample, methods known to one of ordinary skill in the art were used to extract and quantify transcribed RNA from a sample with respect to a constituent of a Gene Expression Panel (Precision Profile™). (See detailed protocols below. Also see PCT application publication number WO 98/24935 herein incorporated by reference for RNA analysis protocols). Briefly, RNA is extracted from a sample such as any tissue (e.g., OA tissue, body fluid, cell, or culture medium in which a population of cells of a subject might be growing. For example, cells may be lysed and RNA eluted in a suitable solution in which to conduct a DNAse reaction. Subsequent to RNA extraction, first strand synthesis may be performed using a reverse transcriptase. Gene amplification, more specifically quantitative PCR assays, can then be conducted and the gene of interest calibrated against an internal marker such as 18S rRNA (Hirayama et al., Blood 92, 1998: 46-52). Any other endogenous marker can be used, such as 28S-25S rRNA and 5S rRNA. Samples are measured in multiple replicates, for example, 3 replicates. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, quantitation of the reporter signal for an internal marker generated by the exponential increase of amplified product may also be used. Amplification of the target template may be accomplished by isothermic gene amplification strategies or by gene amplification by thermal cycling such as PCR.

It is desirable to obtain a definable and reproducible correlation between the amplified target or reporter signal, i.e., internal marker, and the concentration of starting templates. It has been discovered that this objective can be achieved by careful attention to, for example, consistent primer-template ratios and a strict adherence to a narrow permissible level of experimental amplification efficiencies (for example 90.0 to 100%+/−5% relative efficiency, typically 99.8 to 100% relative efficiency). For example, in determining gene expression levels with regard to a single Gene Expression Profile, it is necessary that all constituents of the panels, including endogenous controls, maintain similar amplification efficiencies, as defined herein, to permit accurate and precise relative measurements for each constituent. Amplification efficiencies are regarded as being “substantially similar”, for the purposes of this description and the following claims, if they differ by no more than approximately 10%, preferably by less than approximately 5%, more preferably by less than approximately 3%, and more preferably by less than approximately 1%. Measurement conditions are regarded as being “substantially repeatable”, for the purposes of this description and the following claims, if they differ by no more than approximately +/−10% coefficient of variation (CV), preferably by less than approximately +/−5% CV, more preferably +/−2% CV. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria directly, in such a manner as to compensate for errors, so that the criteria are satisfied after suitable adjustment of assay results.

In practice, tests are run to assure that these conditions are satisfied. For example, the design of all primer-probe sets are done in house, experimentation is performed to determine which set gives the best performance. Even though primer-probe design can be enhanced using computer techniques known in the art, and notwithstanding common practice, it has been found that experimental validation is still useful. Moreover, in the course of experimental validation, the selected primer-probe combination is associated with a set of features:

The reverse primer should be complementary to the coding DNA strand. In one embodiment, the primer should be located across an intron-exon junction, with not more than four bases of the three-prime end of the reverse primer complementary to the proximal exon. (If more than four bases are complementary, then it would tend to competitively amplify genomic DNA.)

In an embodiment of the invention, the primer probe set should amplify cDNA of less than 110 bases in length and should not amplify, or generate fluorescent signal from, genomic DNA or transcripts or cDNA from related but biologically irrelevant loci.

A suitable target of the selected primer probe is first strand cDNA, which in one embodiment may be prepared from whole blood as follows:

(a) Use of Cell Systems or Whole Blood for Ex Vivo Assessment of a Biological Condition Affected by an Agent.

In one embodiment of the invention, any tissue (e.g., OA tissue), body fluid, or cell(s) may be used for ex vivo assessment of a biological condition affected by an agent. Nucleic acids, RNA and/or DNA are purified from cells, tissues or fluids of the test population of cells or indicator cell lines. RNA is preferentially obtained from the nucleic acid mix using a variety of standard procedures (or RNA Isolation Strategies, pp. 55-104, in RNA Methodologies, A laboratory guide for isolation and characterization, 2nd edition, 1998, Robert E. Farrell, Jr., Ed., Academic Press), in the present using a filter-based RNA isolation system from Ambion (RNAqueous™, Phenol-free Total RNA Isolation Kit, Catalog #1912, version 9908; Austin, Tex.).

In another embodiment of the invention, human blood is obtained by venipuncture and prepared for assay by separating samples for baseline, no exogenous stimulus, and one or more pro-disease stimulus with sufficient volume for at least three time points. Typical pro-inflammatory stimuli that may be used include lipopolysaccharide (LPS), phytohemagglutinin (PHA) heat-killed staphylococci (HKS), carrageean, IL-2 plus toxic shock syndrome toxin-1 (TSST1), or cytokine cocktails, and may be used individually or in combination. The aliquots of heparinized, whole blood are mixed with additional test therapeutic compounds and held at 37° C. in an atmosphere of 5% CO₂ for 30 minutes. Stimulus is added at varying concentrations, mixed and held loosely capped at 37° C. for the prescribed timecourse. At defined time-points, cells are lysed and RNA extracted by various standard means.

In accordance with one procedure, the whole blood assay for Gene Expression Profiles determination is carried out as follows: Human whole blood is drawn into 10 mL Vacutainer tubes with Sodium Heparin. Blood samples are mixed by gently inverting tubes 4-5 times. The blood is used within 10-15 minutes of draw. In the experiments, blood is diluted 2-fold, i.e. per sample per time point, 0.6 mL whole blood+0.6 mL stimulus. The assay medium is prepared and the stimulus added as appropriate.

A quantity (0.6 mL) of whole blood is then added into each 12×75 mm polypropylene tube. 0.6 mL of 2× LPS (from E. coli serotype 0127:B8, Sigma #L3880 or serotype 055, Sigma #L4005, 10 ng/mL, subject to change in different lots) into LPS tubes is added. Next, 0.6 mL assay medium is added to the “control” tubes. The caps are closed tightly. The tubes are inverted 2-3 times to mix samples. Caps are loosened to first stop and the tubes incubated at 37° C., 5% CO₂ for 6 hours. At 6 hours, samples are gently mixed to resuspend blood cells, and 0.15 mL is removed from each tube (using a micropipettor with barrier tip), and transfered to 0.15 mL of lysis buffer and mixed. Lysed samples are extracted using an ABI 6100 Nucleic Acid Prepstation following the manufacturer's recommended protocol.

The samples are then centrifuged for 5 min at 500×g, ambient temperature (IEC centrifuge or equivalent, in microfuge tube adapters in swinging bucket), and as much serum from each tube is removed as possible and discarded. Cell pellets are placed on ice; and RNA extracted as soon as possible using an Ambion RNAqueous kit.

In another embodiment of the invention, subjects are initially either exposed or not-exposed to a pro-disease stimulus, and whole blood is obtained by venipuncture subsequent to the exposure/non-exposure to disease stimulus. In one embodiment, the disease stimulus is photoprovocation. In accordance with this embodiment, UV-light provoked skin reactions (photoprovocation) are induced in subjects afflicted with osteoarthritis (DLE, SOLE, or LET) and Source MDx normal subjects/healthy study volunteers. For example, areas of uninvolved skin on the upper back or extensor aspects of the arms may be irradiated with the minimal tanning dose of UVA (60-100 J/cm²) followed by a miminal erythemal dose of UVB daily for a defined period of time. Whole blood is then obtained from these subjects, after each irradiation, and subsequent defined timepoints (e.g., 24 hours after the last irradiation, then weekly for up to 4 weeks) and assayed for gene expression profiles (as described below), and/or serological or whole blood biomarker responses (percent change from baseline levels over time).

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples (see, for example, RT PCR, Chapter 15 in RNA Methodologies, A laboratory guide for isolation and characterization, 2nd edition, 1998, Robert E. Farrell, Jr., Ed., Academic Press; or Chapter 22 pp. 143-151, RNA isolation and characterization protocols, Methods in molecular biology, Volume 86, 1998, R. Rapley and D. L. Manning Eds., Human Press, or 14 in Statistical refinement of primer design parameters, Chapter 5, pp.55-72, PCR applications: protocols for functional genomics, M. A. Innis, D. H. Gelfand and J. J. Sninsky, Eds., 1999, Academic Press) Amplifications are carried out in either isothermic conditions or using a thermal cycler (for example, a ABI 9600 or 9700 or 7900 obtained from Applied Biosystems, Foster City, Calif.; see Nucleic acid detection methods, pp. 1-24, in Molecular methods for virus detection, D. L. Wiedbrauk and D. H., Farkas, Eds., 1995, Academic Press). Amplified nucleic acids are detected using fluorescent-tagged detection oligonucleotide probes (see, for example, Taqman™ PCR Reagent Kit, Protocol, part number 402823, Revision A, 1996, Applied Biosystems, Foster City Calif.) that are identified and synthesized from publicly known databases as described for the amplification primers. In the present case, amplified cDNA is detected and quantified using the ABI Prism 7900 Sequence Detection System obtained from Applied Biosystems (Foster City, Calif.). Amounts of specific RNAs contained in the test sample or obtained from the indicator cell lines can be related to the relative quantity of fluorescence observed (see for example, Advances in quantitative PCR technology: 5′ nuclease assays, Y. S. Lie and C. J. Petropolus, Current Opinion in Biotechnology, 1998, 9:43-48, or Rapid thermal cycling and PCR kinetics, pp. 211-229, chapter 14 in PCR applications: protocols for functional genomics, M. A. Innis, D. H. Gelfand and J. J. Sninsky, Eds., 1999, Academic Press).

As a particular implementation of the approach described here in detail is a procedure for synthesis of first strand cDNA for use in PCR. This procedure can be used for both whole blood RNA and RNA extracted from cultured cells.

Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent)

Methods

1. Place RNase Inhibitor and MultiScribe Reverse Transcriptase on ice immediately. All other reagents can be thawed at room temperature and then placed on ice.

2. Remove RNA samples from −80° C. freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| | |
|---|---|
| | 11X, e.g. |
| 1 reaction (mL) | 10 samples (μL) |

| | | |
|---|---|---|
| 10X RT Buffer | 10.0 | 110.0 |
| 25 mM MgCl₂ | 22.0 | 242.0 |
| dNTPs | 20.0 | 220.0 |
| Random Hexamers | 5.0 | 55.0 |
| RNAse Inhibitor | 2.0 | 22.0 |
| Reverse Transcriptase | 2.5 | 27.5 |
| Water | 18.5 | 203.5 |
| Total: | 80.0 | 880.0 |
| | | (80 μL per sample) |

4. Bring each RNA sample to a total volume of 20 μL in a 1.5 mL microcentrifuge tube (for example, for THP-1 RNA, remove 10 μL RNA and dilute to 20 μL with RNase/DNase free water, for whole blood RNA use 20 μL total RNA) and add 80 μL RT reaction mix from step 5,2,3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and β-actin.

The use of the primer probe with the first strand cDNA as described above to permit measurement of constituents of a Gene Expression Panel (Precision Profile™) is as follows:

Materials

1. 20× Primer/Probe Mix for each gene of interest.

2. 20× Primer/Probe Mix for 18S endogenous control.

3. 2× Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism 7700 or 7900 Sequence Detector.

Methods

1. Make stocks of each Primer/Probe mix containing the Primer/Probe for the gene of interest, Primer/Probe for 18S endogenous control, and 2× PCR Master Mix as follows. Make sufficient excess to allow for pipetting error e.g., approximately 10% excess. The following example illustrates a typical set up for one gene with quadruplicate samples testing two conditions (2 plates).

| | 1X (1 well) (μL) |
|---|---|
| 2X Master Mix | 7.5 |
| 20X 18S Primer/Probe Mix | 0.75 |
| 20X Gene of interest Primer/Probe Mix | 0.75 |
| Total | 9.0 |

2. Make stocks of cDNA targets by diluting 95 μL of cDNA into 2000 μL of water. The amount of cDNA is adjusted to give Ct values between 10 and 18, typically between 12 and 16.

3. Pipette 9 μL of Primer/Probe mix into the appropriate wells of an Applied Biosystems 384-Well Optical Reaction Plate.

4. Pipette 10 μL of cDNA stock solution into each well of the Applied Biosystems 384-Well Optical Reaction Plate.

5. Seal the plate with Applied Biosystems Optical Caps, or optical-clear film.

6. Analyze the plate on the ABI Prism 7900 Sequence Detector.

In another embodiment of the invention, the use of the primer probe with the first strand cDNA as described above to permit measurement of constituents of a Gene Expression Panel (Precision Profile™) is performed using a QPCR assay on Cepheid SmartCycler® and GeneXpert® Instruments as follows:

I. To run a QPCR assay in duplicate on the Cepheid SmartCycler® instrument containing three target genes and one reference gene, the following procedure should be followed.

A. With 20× Primer/Probe Stocks.

### Materials

1. SmartMix™-HM lyophilized Master Mix.
2. Molecular grade water.
3. 20× Primer/Probe Mix for the 18S endogenous control gene. The endogenous control gene will be dual labeled with VIC-MGB or equivalent.
4. 20× Primer/Probe Mix for each for target gene one, dual labeled with FAM-BHQ1 or equivalent.
5. 20× Primer/Probe Mix for each for target gene two, dual labeled with Texas Red-BHQ2 or equivalent.
6. 20× Primer/Probe Mix for each for target gene three, dual labeled with Alexa 647-BHQ3 or equivalent.
7. Tris buffer, pH 9.0
8. cDNA transcribed from RNA extracted from sample.
9. SmartCycler® 25 μL tube.
10. Cepheid SmartCycler® instrument.

### Methods

1. For each cDNA sample to be investigated, add the following to a sterile 650 μL tube.

| | | |
|---|---|---|
| SmartMix ™-HM lyophilized Master Mix | 1 | bead |
| 20X 18S Primer/Probe Mix | 2.5 | μL |
| 20X Target Gene 1 Primer/Probe Mix | 2.5 | μL |
| 20X Target Gene 2 Primer/Probe Mix | 2.5 | μL |
| 20X Target Gene 3 Primer/Probe Mix | 2.5 | μL |
| Tris Buffer, pH 9.0 | 2.5 | μL |
| Sterile Water | 34.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.

B. With Lyophilized SmartBeads™.

### Materials

1. SmartMix™-HM lyophilized Master Mix.
2. Molecular grade water.
3. SmartBeads™ containing the 18S endogenous control gene dual labeled with VIC-MGB or equivalent, and the three target genes, one dual labeled with FAM-BHQ1 or equivalent, one dual labeled with Texas Red-BHQ2 or equivalent and one dual labeled with Alexa 647-BHQ3 or equivalent.
4. Tris buffer, pH 9.0
5. cDNA transcribed from RNA extracted from sample.
6. SmartCycler® 25 μL tube.
7. Cepheid SmartCycler® instrument.

### Methods

1. For each cDNA sample to be investigated, add the following to a sterile 650 μL tube.

| | | |
|---|---|---|
| SmartMix ™-HM lyophilized Master Mix | 1 | bead |
| SmartBead ™ containing four primer/probe sets | 1 | bead |
| Tris Buffer, pH 9.0 | 2.5 | μL |
| Sterile Water | 44.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.

II. To run a QPCR assay on the Cepheid GeneXpert® instrument containing three target genes and one reference gene, the following procedure should be followed. Note that to do duplicates, two self contained cartridges need to be loaded and run on the GeneXpert® instrument.

### Materials

1. Cepheid GeneXpert® self contained cartridge preloaded with a lyophilized SmartMix™-HM master mix bead and a lyophilized SmartBead™ containing four primer/probe sets.
2. Molecular grade water, containing Tris buffer, pH 9.0.
3. Extraction and purification reagents.
4. Clinical sample (whole blood, RNA, etc.)
5. Cepheid GeneXpert® instrument.

### Methods

1. Remove appropriate GeneXpert® self contained cartridge from packaging.
2. Fill appropriate chamber of self contained cartridge with molecular grade water with Tris buffer, pH 9.0.
3. Fill appropriate chambers of self contained cartridge with extraction and purification reagents.
4. Load aliquot of clinical sample into appropriate chamber of self contained cartridge.
5. Seal cartridge and load into GeneXpert® instrument.
6. Run the appropriate extraction and amplification protocol on the GeneXpert® and analyze the resultant data.

Methods herein may also be applied using proteins where sensitive quantitative techniques, such as an Enzyme Linked ImmunoSorbent Assay (ELISA) or mass spectroscopy, are available and well-known in the art for measuring the amount of a protein constituent. (see WO 98/24935 herein incorporated by reference).

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a biological condition or agent. Baseline profile data sets may be stored in libraries and classified in a number of cross-referential ways. One form of classification may rely on the characteristics of the panels from which the data sets are derived. Another form of classification may be by particular biological condition, e.g., osteoarthritis. The concept of biological condition encompasses any state in which a cell or population of cells may be found at any one time. This state may reflect geography of samples, sex of subjects or any other discriminator. Some of the discriminators may overlap. The libraries may also be accessed for records associated with a single subject or particular clinical trial. The classification of baseline profile data sets may further be annotated with medical information about a particular subject, a medical condition, and/or a particular agent.

The choice of a baseline profile data set for creating a calibrated profile data set is related to the biological condition to be evaluated, monitored, or predicted, as well as, the intended use of the calibrated panel, e.g., as to monitor drug development, quality control or other uses. It may be desirable to access baseline profile data sets from the same subject for whom a first profile data set is obtained or from different subject at varying times, exposures to stimuli, drugs or complex compounds; or may be derived from like or dissimilar populations or sets of subjects. The baseline profile data set may be normal, healthy baseline.

The profile data set may arise from the same subject for which the first data set is obtained, where the sample is taken at a separate or similar time, a different or similar site or in a different or similar biological condition. For example, a sample may be taken before stimulation or after stimulation with an exogenous compound or substance, such as before or after therapeutic treatment. Alternatively the sample is taken before or include before or after a surgical procedure for osteoarthritis. The profile data set obtained from the unstimulated sample may serve as a baseline profile data set for the sample taken after stimulation. The baseline data set may also be derived from a library containing profile data sets of a population or set of subjects having some defining characteristic or biological condition. The baseline profile data set may also correspond to some ex vivo or in vitro properties associated with an in vitro cell culture. The resultant calibrated profile data sets may then be stored as a record in a database or library along with or separate from the baseline profile data base and optionally the first profile data set although the first profile data set would normally become incorporated into a baseline profile data set under suitable classification criteria. The remarkable consistency of Gene Expression Profiles associated with a given biological condition makes it valuable to store profile data, which can be used, among other things for normative reference purposes. The normative reference can serve to indicate the degree to which a subject conforms to a given biological condition (healthy or diseased) and, alternatively or in addition, to provide a target for clinical intervention.

Selected baseline profile data sets may be also be used as a standard by which to judge manufacturing lots in terms of efficacy, toxicity, etc. Where the effect of a therapeutic agent is being measured, the baseline data set may correspond to Gene Expression Profiles taken before administration of the agent. Where quality control for a newly manufactured product is being determined, the baseline data set may correspond with a gold standard for that product. However, any suitable normalization techniques may be employed. For example, an average baseline profile data set is obtained from authentic material of a naturally grown herbal nutraceutical and compared over time and over different lots in order to demonstrate consistency, or lack of consistency, in lots of compounds prepared for release.

### Calibrated Data

Given the repeatability achieved in measurement of gene expression, described above in connection with “Gene Expression Panels” (Precision Profiles™) and “gene amplification”, it was concluded that where differences occur in measurement under such conditions, the differences are attributable to differences in biological condition. Thus, it has been found that calibrated profile data sets are highly reproducible in samples taken from the same individual under the same conditions. Similarly, it has been found that calibrated profile data sets are reproducible in samples that are repeatedly tested. Also found have been repeated instances wherein calibrated profile data sets obtained when samples from a subject are exposed ex vivo to a compound are comparable to calibrated profile data from a sample that has been exposed to a sample in vivo. Importantly, it has been determined that an indicator cell line treated with an agent can in many cases provide calibrated profile data sets comparable to those obtained from in vivo or ex vivo populations of cells. Moreover, it has been determined that administering a sample from a subject onto indicator cells can provide informative calibrated profile data sets with respect to the biological condition of the subject including the health, disease states, therapeutic interventions, aging or exposure to environmental stimuli or toxins of the subject. Thus, the Precision Profiles of the invention are fully calibrated, allowing for direct comparisons of expression levels of individual genes in a panel. This calibration is critical in developing data that can be used to develop, test and refine biomedical algorithms and models.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within one order of magnitude with respect to similar samples taken from the subject under similar conditions. More particularly, the members may be reproducible within 20%, and typically within 10%. In accordance with embodiments of the invention, a pattern of increasing, decreasing and no change in relative gene expression from each of a plurality of gene loci examined in the Gene Expression Panel (Precision Profile™) may be used to prepare a calibrated profile set that is informative with regards to a biological condition, biological efficacy of an agent treatment conditions or for comparison to populations or sets of subjects or samples, or for comparison to populations of cells. Patterns of this nature may be used to identify likely candidates for a drug trial, used alone or in combination with other clinical indicators to be diagnostic or prognostic with respect to a biological condition or may be used to guide the development of a pharmaceutical or nutraceutical through manufacture, testing and marketing.

The numerical data obtained from quantitative gene expression and numerical data from calibrated gene expression relative to a baseline profile data set may be stored in databases or digital storage mediums and may be retrieved for purposes including managing patient health care or for conducting clinical trials or for characterizing a drug. The data may be transferred in physical or wireless networks via the World Wide Web, email, or internet access site for example or by hard copy so as to be collected and pooled from distant geographic sites.

The method also includes producing a calibrated profile data set for the panel, wherein each member of the calibrated profile data set is a function of a corresponding member of the first profile data set and a corresponding member of a baseline profile data set for the panel, and wherein the baseline profile data set is related to the osteoarthritis or conditions related to osteoarthritis to be evaluated, with the calibrated profile data set being a comparison between the first profile data set and the baseline profile data set, thereby providing evaluation of osteoarthritis or conditions related to osteoarthritis of the subject.

In yet other embodiments, the function is a mathematical function and is other than a simple difference, including a second function of the ratio of the corresponding member of first profile data set to the corresponding member of the baseline profile data set, or a logarithmic function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data. Because the data is in a universal format, data handling may readily be done with a computer. The data is organized so as to provide an output optionally corresponding to a graphical representation of a calibrated data set.

For example, a distinct sample derived from a subject being at least one of RNA or protein may be denoted as PI. The first profile data set derived from sample PI is denoted Mj, where Mj is a quantitative measure of a distinct RNA or protein constituent of PI. The record Ri is a ratio of M and P and may be annotated with additional data on the subject relating to, for example, age, diet, ethnicity, gender, geographic location, medical disorder, mental disorder, medication, physical activity, body mass and environmental exposure. Moreover, data handling may further include accessing data from a second condition database which may contain additional medical data not presently held with the calibrated profile data sets. In this context, data access may be via a computer network.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The graphical representation of calibrated profile data sets pertaining to a product such as a drug provides an opportunity for standardizing a product by means of the calibrated profile, more particularly a signature profile. The profile may be used as a feature with which to demonstrate relative efficacy, differences in mechanisms of actions, etc. compared to other drugs approved for similar or different uses.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set. The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

In other embodiments, a clinical indicator may be used to assess the osteoarthritis or conditions related to osteoarthritis of the relevant set of subjects by interpreting the calibrated profile data set in the context of at least one other clinical indicator, wherein the at least one other clinical indicator is selected from the group consisting of blood chemistry, molecular markers in the blood (e.g., positive or negative titer from anti-nuclear antibody test or anti-RO (SSA), other chemical assays, and physical findings.

### Index Construction

In combination, (i) the remarkable consistency of Gene Expression Profiles with respect to a biological condition across a population or set of subject or samples, or across a population of cells and (ii) the use of procedures that provide substantially reproducible measurement of constituents in a Gene Expression Panel (Precision Profile™) giving rise to a Gene Expression Profile, under measurement conditions wherein specificity and efficiencies of amplification for all constituents of the panel are substantially similar, make possible the use of an index that characterizes a Gene Expression Profile, and which therefore provides a measurement of a biological condition.

An index may be constructed using an index function that maps values in a Gene Expression Profile into a single value that is pertinent to the biological condition at hand. The values in a Gene Expression Profile are the amounts of each constituent of the Gene Expression Panel (Precision Profile™) that corresponds to the Gene Expression Profile. These constituent amounts form a profile data set, and the index function generates a single value—the index—from the members of the profile data set.

The index function may conveniently be constructed as a linear sum of terms, each term being what is referred to herein as a “contribution function” of a member of the profile data set. For example, the contribution function may be a constant times a power of a member of the profile data set. So the index function would have the form

I=ΣCiMi^{P(i)},

where I is the index, Mi is the value of the member i of the profile data set, Ci is a constant, and P(i) is a power to which Mi is raised, the sum being formed for all integral values of i up to the number of members in the data set. We thus have a linear polynomial expression. The role of the coefficient Ci for a particular gene expression specifies whether a higher ACt value for this gene either increases (a positive Ci) or decreases (a lower value) the likelihood of osteoarthritis, the ACt values of all other genes in the expression being held constant.

The values Ci and P(i) may be determined in a number of ways, so that the index I is informative of the pertinent biological condition. One way is to apply statistical techniques, such as latent class modeling, to the profile data sets to correlate clinical data or experimentally derived data, or other data pertinent to the biological condition. In this connection, for example, may be employed the software from Statistical Innovations, Belmont, Massachusetts, called Latent Gold®. Alternatively, other simpler modeling techniques may be employed in a manner known in the art. The index function for osteoarthritis may be constructed, for example, in a manner that a greater degree of osteoarthritis (as determined by the profile data set for the Precision Profile™ for Osteoarthritis shown in Table 1 or Precision Profile™ for Inflammatory Response shown in Table 2) correlates with a large value of the index function. As discussed in further detail below, a meaningful osteoarthritis index that is proportional to the expression, was constructed as follows:

LOGIT=22.97−0.60 {PF4}−{IL6R}

where the braces around a constituent designate measurement of such constituent and the constituents are a subset of the Precision Profile™ for Osteoarthritis shown in Table 1 or Precision Profile™ for Inflammatory Response shown in Table 2.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition, clinical indicator, medication, physical activity, body mass, and environmental exposure.

As an example, the index can be constructed, in relation to a normative Gene Expression Profile for a population or set of healthy subjects, in such a way that a reading of approximately 1 characterizes normative Gene Expression Profiles of healthy subjects. Let us further assume that the biological condition that is the subject of the index is osteoarthritis; a reading of 1 in this example thus corresponds to a Gene Expression Profile that matches the norm for healthy subjects. A substantially higher reading then may identify a subject experiencing osteoarthritis, or a condition related to osteoarthritis. The use of 1 as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since it was determined that Gene Expression Profile values (and accordingly constructed indices based on them) tend to be normally distributed, the 0-centered index constructed in this manner is highly informative. It therefore facilitates use of the index in diagnosis of disease and setting objectives for treatment.

Still another embodiment is a method of providing an index pertinent to osteoarthritis or conditions related to osteoarthritis of a subject based on a first sample from the subject, the first sample providing a source of RNAs, the method comprising deriving from the first sample a profile data set, the profile data set including a plurality of members, each member being a quantitative measure of the amount of a distinct RNA constituent in a panel of constituents selected so that measurement of the constituents is indicative of the presumptive signs of osteoarthritis, the panel including at least two of the constituents of any of the genes listed in the Precision Profile for Osteoarthritis™ (Table 1) or the Precision Profile™ for Inflammatory Response (Table 2). In deriving the profile data set, such measure for each constituent is achieved under measurement conditions that are substantially repeatable, at least one measure from the profile data set is applied to an index function that provides a mapping from at least one measure of the profile data set into one measure of the presumptive signs of osteoarthritis, so as to produce an index pertinent to the osteoarthritis or conditions related to osteoarthritis of the subject.

As another embodiment of the invention, an index function I of the form

I=C₀+ΣCᵢMₗᵢ^{P1(i)} M₂ᵢP2(i),

can be employed, where M₁ and M₂ are values of the member i of the profile data set, C, is a constant determined without reference to the profile data set, and P1 and P2 are powers to which M₁ and M₂ are raised. The role of P1(i) and P2(i) is to specificy the specific functional form of the quadratic expression, whether in fact the equation is linear, quadratic, contains cross-product terms, or is constant. For example, when P1=P2=0, the index function is simply the sum of constants; when P1=1 and P2=0, the index function is a linear expression; when P1=P2=1, the index function is a quadratic expression.

The constant C₀ serves to calibrate this expression to the biological population of interest that is characterized by having osteoarthritis. In this embodiment, when the index value equals 0, the odds are 50:50 of the subject having osteoarthritis vs a normal subject. More generally, the predicted odds of the subject having osteoarthritis is [exp(I,)], and therefore the predicted probability of having osteoarthritis is [exp(Iᵢ)]/[1+exp((Iᵢ)]. Thus, when the index exceeds 0, the predicted probability that a subject has osteoarthritis is higher than 0.5, and when it falls below 0, the predicted probability is less than 0.5.

The value of C₀ may be adjusted to reflect the prior probability of being in this population based on known exogenous risk factors for the subject. In an embodiment where C₀ is adjusted as a function of the subject's risk factors, where the subject has prior probability pᵢ of having osteoarthritis based on such risk factors, the adjustment is made by increasing (decreasing) the unadjusted C₀ value by adding to C₀ the natural logarithm of the ratio of the prior odds of having osteoarthritis taking into account the risk factors to the overall prior odds of having osteoarthritis without taking into account the risk factors.

### Kits

The invention also includes an osteoarthritis detection reagent, i.e., nucleic acids that specifically identify one or more osteoarthritis or condition related to osteoarthritis nucleic acids (e.g., any gene listed in Tables 1-2, 4-6, and 8; sometimes referred to herein as osteoarthritis associated genes or osteoarthritis associated constituents) by having homologous nucleic acid sequences, such as oligonucleotide sequences, complementary to a portion of the osteoarthritis genes nucleic acids or antibodies to proteins encoded by the osteoarthritis genes nucleic acids packaged together in the form of a kit. The oligonucleotides can be fragments of the osteoarthritis genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label. Instructions (i.e., written, tape, VCR, CD-ROM, etc.) for carrying out the assay may be included in the kit. The assay may for example be in the form of PCR, a Northern hybridization or a sandwich ELISA, as known in the art.

For example, osteoarthritis genes detection reagents can be immobilized on a solid matrix such as a porous strip to form at least one osteoarthritis gene detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of osteoarthritis genes present in the sample. The detection sites may be configured in any suitably detectable shape and are typically in the shape of a bar or dot spanning the width of a test strip.

Alternatively, osteoarthritis detection genes can be labeled (e.g., with one or more fluorescent dyes) and immobilized on lyophilized beads to form at least one osteoarthritis gene detection site. The beads may also contain sites for negative and/or positive controls. Upon addition of the test sample, the number of sites displaying a detectable signal provides a quantitative indication of the amount of osteoarthritis genes present in the sample.

Alternatively, the kit contains a nucleic acid substrate array comprising one or more nucleic acid sequences. The nucleic acids on the array specifically identify one or more nucleic acid sequences represented by osteoarthritis genes (see Tables 1-2, 4-6, and 8). In various embodiments, the expression of 1, 2, 3, 4, 5, 6, 7, 8, 9, 10, 15, 20, 25, 40 or 50 or more of the sequences represented by osteoarthritis genes (see Tables 1-2, 4-6, and 8) can be identified by virtue of binding to the array. The substrate array can be on, i.e., a solid substrate, i.e., a “chip” as described in U.S. Pat. No. 5,744,305. Alternatively, the substrate array can be a solution array, i.e., Luminex, Cyvera, Vitra and Quantum Dots' Mosaic.

The skilled artisan can routinely make antibodies, nucleic acid probes, i.e., oligonucleotides, aptamers, siRNAs, antisense oligonucleotides, against any of the osteoarthritis genes listed in Tables 1-2, 4-6, and 8.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1

### Clinical and Pathological Assessment of Subjects Suffering from Osteoarthritis

This study involves detailed clinical and pathological assessments of the participants' knee osteoarthritis severity including a measure of cartilage voume loss during a two-year observation period, determined using 3-dimensional MRI. The procedures are summarized in Table 3. Blood for gene expression anlysis are collected into PAXGENE® tubes at all timepoints.

Inclusion Criteria for the subjects in this study are as follows: age: >49 years, chronic knee discomfort based on affirmative response to the question “During the past 12 months, have you had any pain aching or stiffness in your knees?”, WOMAC pain subscale score ≧1, Tibiofemoral or patellofemoral OA on anteroposterior weight-bearing semi-flexed or lateral knee radiographs with severity equivalent to Kellgren and Lawrence grade >=2 (i.e. at least one osteophyte >=grade 2 on the Osteoarthritis Research Society standard atlas¹⁰⁰), clinical examination confirming knee pain or discomfort referable to the knee joint, and prepared to refrain from use of glucosamine, chondroitin, diacerein and doxycycline.

Exclusion Criteria for the subjects in this study are as follows: serum 25(OH) vitamin D level >80 ng/ml, use of glucosamine, chondroitin, diacerein or doxycycline within three months, hypercalcemia (>10.5 mg/dL), evidence of vitamin D toxicity through abnormal values according to laboratory reference standards for calcium, 25(OH)D or parathormone, history of lymphoma, or sarcoidosis. Currently on treatment for tuberculosis, serious medical conditions or impairments that, in the view of the investigator, would obstruct their participation in the trial, plan to permanently relocate from the region during the trial period, planned knee arthroplasty in the study knee, and any contra-indication to having an MRI scan (pacemaker; intracranial clip; aneurysm clip; metallic heart valve prosthesis; metallic object in the eye from an accident; shrapnel/other metal in body; dentures, retainer, braces; coronary artery bypass clip; renal transplant clips; other vascular clips; metal I.U.D.; middle ear prosthesis; hearing aid; wig; limb or joint prosthesis; orbital prosthesis; transcutaneous nerve stimulator; biostimulator; and insulin pump).

### Clinical Outcome Assessments

### The WOMAC Osteoarthritis Index

The Western Ontario and McMaster Universities (WOMAC) osteoarthritis index is a tri-dimensional disease-specific self-administered health status questionnaire. It probes clinically important, patient relevant symptoms in the areas of pain, stiffness and physical function in patients with OA of the hip or knee. The index consists of 24 questions (5 pain, 2 stiffness, 17 physical function) which can be completed by the patient in 5 minutes. WOMAC has high test retest reliability for all scales, and validation studies have showed high correlations with other indices probing the same dimensions including MHIQ, Doyle, the Lequesne index and others. Responsiveness has been tested in non-steroidal trials and each aggregated subscale score (e.g. pain) has been found to detect the effect of NSAID's101, and to detect a clinically important statistically significant difference in efficacy between two NSAIDs. In terms of sensitivity to change, WOMAC has been compared to other measures of patient status in OA including HAQ, AIMS, the Doyle index the Lequesne index and measures of walk time, range of motion, and has generally been found to be more sensitive to change (relative efficiency compared to other instruments ≧1). It can be utilized in a site-specific fashion and has been shown to discriminate between outcomes in opposite joints in the same patients108. The WOMAC has been recommended as a measure for assessing ‘slow-acting’ drugs in OA, and has been employed in two recently completed three year clinical trials of glucosamine for knee OA that had positive results using this instrument.

Bellamy et al. have also developed, tested and validated a computerized version of the WOMAC visual analog scale instrument. The computerized instrument was depicted in a format very similar to the original version, with visual analog scales and cursors which could be moved by the mouse. Numeric values between 0 and 100 were generated corresponding to the placement of the cursor. The instrument was found to be easy to use, with participants completing the questionnaire within 15 minutes. Concordance with scores assigned on the paper instrument was excellent, as was criterion validity based on aggregated subscale scores.

### Physical Function Tests

To obtain objective measures of lower extremity physical function a short battery of standardized physical performance tests is adminstered, which have been validated and widely used, including among individuals with knee pain. Time to walk is evaluated using a stopwatch and a measured 6-meter course. In this test the participant is instructed to walk at a normal pace while the observer measures the time in seconds. The test is performed twice. Strength and endurance is tested by counting the number of times that the participant can fully rise and sit from a chair without using their arms during a 15 second period. Since disturbance of balance is common in older people, and an important contributor to lower extremity functional impairment, this is tested by examining ability to stand with the feet together in the side-by-side, semi-tandem, and tandem positions.

### SF-36® Health Survey

The SF-36® Health Survey is a multi-purpose, health-related quality of life survey with only 36 questions118. It yields an 8-scale profile of functional health and well-being scores as well as psychometrically based physical and mental health summary measures and a preference-based health utility index. It is a generic health measure, as opposed to one that targets a specific age, disease, or treatment group. However, it has been widely used in rheumatic disease trials, and has been validated in patients with osteoarthritis and rheumatoid arthritis.

### Analgesic Requirement

In the course of a two-year trial, it is likely that participants will consume a variety of non-steroidal anti-inflammatory agents and analgesics. Information regarding all analgesics and nutriceuticals taken by the subjects during the course of the trial for their knee(s) is collected. Each participant is provided with a paper calendar to enable them to keep a record that they can produce at each visit. In order to make quantitative comparisons of the different analgesics used by trial participants, consumption of each analgesic is converted into acetaminophen equivalents based on published comparative data.

### Arthroplasty

During the two year trial period, any knee arthroplasties that take place is recorded. While it is unlikely that large numbers of these will occur during the course of the trial, this information will complement our overall outcome assessment for individuals, especially as progression in both knees is being evaluated over the trial period.

### MRI Outcome Assessments

### MRI Scanner

MRI scans of each participant's study knee at baseline, one-year and at the final (year 2) visit are obtained using a Siemens Aventa 1.5T scanner. A dedicated circularly polarized transmit-receive lower extremity coil is available for knee imaging. The upper part of the coil can be removed for easy patient/subject positioning. In addition, because of the circular polarization and high filling factor for the knee, this coil is ideal for high resolution imaging of the knee with excellent signal/noise ratio.

After standard “localizer” pulse sequences are run to determine the volume of interest, the following dedicated sequences for quantitative and semi-quantitative assessment of knee OA are obtained;

(1) A 3-dimensional double-echo steady-state MR sequence in the sagittal plane (TR=27.5 msec, TE=9.0 msec, flip angle 30°, 1 excitation (NEX), matrix 512×256 elements, FOV 11×11 cm, slice thickness 1.3 mm, 52 slices). Excellent spatial resolution, contrast-to-noise ratios, and precision can be achieved using these parameters (see Preliminary Results, C.i). This sequence is used to render the cartilage in three dimensions.

(2) Proton-density (PD)-weighted fast spin-echo sequence in sagittal plane (TR=3000 msec, TE=17 msec, 2 excitation (NEX), matrix elements 256×256, FOV 14 cm, slice thickness 3 mm, 26 slices).

(3) T2-weighted fast spin-echo fat-suppressed sequence in sagittal plane (TR=2500 msec, TE=76 msec, 1 excitation (NEX), matrix elements 256×256, FOV 14 cm, slice thickness 3 mm, 26 slices).

(4) Proton-density (PD)-weighted fast spin-echo sequence in coronal plane (TR=3000 msec, TE=15 msec, 1 excitation (NEX), matrix elements 256×256 , FOV 15 cm, slice thickness 3 mm, 24 slices).

(5) T2-weighted fast spin-echo fat-suppressed sequence in coronal plane (TR=4100 msec, TE=76 msec, 1 excitation (NEX), matrix elements 256×256, FOV 15 cm, slice thickness 3 mm, 24 slices).

### MR Image Processing

The fat saturated 3D gradient echo MR images of the knee is transferred via Ethernet to an independent computer workstation running ANALYZE software analysis package (Biomedical Imaging Resource, Mayo Clinic, Rochester, Minn.). ANALYZE has a DICOM query/retrieve feature that allows direct interrogation of the MRI scanner database over the network and subsequent retrieval of selected data. Before segmentation of the cartilage takes place, an algorithm is used to correct for any B1 RF field inhomogeneities from the extremity RF coil used to acquire the data. There is a built-in tool within ANALYZE to accomplish this. To calculate the inhomogeneity correction, ANALYZE first uses a low pass spatial filter with a kernel size of 64×64 voxels to obtain images with all the fine structure removed. This is then used to calculate a correction such that this variation is then corrected on the original images. After B1 correction, cartilage segmentation will be performed. For this, the Region of Interest Tool together with a seeded, region-growing algorithm based on a dual image threshold (lower and upper image intensity specified) is used. Once the thresholds are set for a particular slice manually, these thresholds are automatically transferred to each successive slice. Since the signal intensity of the cartilage is ideally not a function of slice, the operator then only needs to make minor adjustments in the thresholds of successive slices to define the cartilage boundary. If the cartilage is damaged or if there are unconnected regions of a particular cartilage, then additional seeds arebused to define more than one region associated with a particular cartilage. Within ANALYZE, these disconnected regions are assigned to the same object class so that can be treated properly. Different types of cartilage (i.e. patellar, femoral, tibial) are assigned to different classes. After segmentation, an OBJECT map is created with pixels defined as being part of or not part of different structures (or classes). Statistics (volume, mean intensity, surface area, standard deviation of pixel intensity, etc.) for each cartilage class within the 3D volume are easily computed with another tool within ANALYZE. 3D maps of cartilage thickness are then easily generated using the volume rendering tool with ANALYZE.

### MRI Whole Knee OA Severity Semi-Quantitative Scale

Cartilage loss is graded in the anterior, central, posterior regions of the medial and lateral knee compartments on a scale from 0-6 [normal=grade 0, signal heterogeneity (focal or diffuse signal heterogeneity with an intact cartilage surface=grade 1, superficial fraying=grade 2, fissuring=grade 3, thinning less than 50%=grade 4, thinning greater than 50%=grade 5, and full thickness cartilage loss=grade 6. The size of the lesion is also scored: lesions measuring less than or equal to 1 cm² grade ‘A’, lesions 1-2 cm² grade ‘B’, lesions 2-3 cm² grade ‘C’, lesions 3-5 cm² grade ‘D’, lesions >5 cm² grade ‘E’.

Meniscal and cruciate ligament pathology is also evaluated. Bone marrow edema is graded none (grade=0), mild (extending less than 1 cm from the subchondral bone, grade=1), moderate (extending 1-2 cm from the subchondral bone, grade=2) and severe (extending greater than 2 cm from the subchondral bone, grade=3). Osteophytes, subchondral cysts and subchondral sclerosis are also graded on a 0-3 scale.

### Reader Training and Certification Protocol

At the outset of the study, a reader certification set of twenty knee MRI scans selected to represent the range of OA severity is assembled. The radiologist readers sit together and practice scoring a set of training images to achieve familiarity and standardization in its application. Each radiologist reader independently scores the set of certification MRI scans using the technical description [Peterfy, 2004 #1436] as a gold standard. Their inter-observer agreement will be evaluated. If the inter-observer agreement values are not comparable to those found in the technical description (most ICCs >0.8), the radiologist readers are retrained.

### Reproducibility and Quality Control Of MRI Outcome Measures

All images are graded twice, with the reader blinded to the identity and sequence of the images. The test-retest and intra-rater reliability, and variance, of each MRI scoring system is computed. Test-retest and intra-rater reliability is evaluated using the paired MRI readings on each subject. Measurement ‘drift’ over time by re-presenting a core set of images to each reader at quasi-random time points in a covert fashion is evaluated. An additional quality control procedure is to send three sets of twenty MRI scans drawn from the beginning, middle and end of the trial to expert radiology reader(s). The expert reader(s) perform independent measurements on these images. The scores of the experts are compared to the scores generated within the study and the basis of any differences to improve the validity and reliability of our own assessments is examined. The expert reader also visits impose initial quality surveillance at the start of cartilage volume measurement activities. For continuous outcomes, interclass correlation coefficients are computed and the method of Bland and Altman are used to determine if reliability is affected by the outcome value. For the ordinal scales, weighted kappa statistics are computed.

### Example 2

### Clinical Data Analyzed with Latent Class Modeling (1 and 2-Gene Models) Based on an Osteoarthritis Gene Expression Panel

RNA Extraction and preparation of cDNA

Whole blood samples for gene-expression analysis were collected from a total of 40 subjects suffering from symptomatic knee osteoarthritis and 40 normal subjects and placed directly into PAXgene® tubes (PreAnalytiX) to stabilize gene activity. These tubes contain proprietary additives that effectively inhibit RNase-mediated degradation activities and prevent activation of gene transcription that may occur as a result of phlebotomy. Samples were frozen within 24 hours of collection to permit batch preparation and analysis. RNA was extracted from the whole blood samples using the PAXgene accompanying extraction chemistry and procedures (PAXgene® Blood RNA Kit). RNA samples were treated with RNase-Free DNase I using manufacturer recommended protocols during the purification process, for digestion of contaminating genomic DNA.

To quality control test total RNA, the Total RNA Quantitative Measurement was used to determine the concentration of total RNA in each extracted PAXgene® Blood RNA Tube sample. The Bioanalyzer 2100 (Agilent Technologies) in combination with the RNA 6000 LabChip, was used for this evaluation. The RNA concentration from extracted PAXgene samples must be within a defined concentration range in order to proceed with first strand synthesis. In addition, the Total RNA Quality Assessment determines the integrity of extracted RNA from each PAXgene® Blood RNA Tube sample. RNA integrity was visualized with electropherograms and gel-like images produced using the Bioanalyzer 2100 (Agilent Technologies) in combination with the RNA 6000 LabChip. The ratio of the peak areas for the 18S/28S ribosomal bands for all samples was calculated. Variability in this ratio may indicate partial degradation of the sample during the purification procedure. This information, along with the separation analysis, gave an indication of the the quality of the RNA preparation. In addition, the purity of the RNA sample was also determined by the presence or absence of genomic DNA contaminants visualized on the electropherogram.

First-strand cDNA was synthesized by reverse transcription following priming with random hexamers, using TaqMan® Reverse Transcription reagents (Applied Biosystems) and an ABI Prism 6700 robot. To quality control test the cDNA, an 18S rRNA Quantitative Measurement was used to determine the quantity of cDNA first strand template synthesized from purified RNA samples. It was imperative that quantitative PCR (QPCR) analysis of thel8S rRNA content of newly synthesized cDNA template, using the ABI Prism® 7900 Sequence Detection System, be within a defined range of values for subsequent use in QPCR analysis of specified target genes. In addition, 18S rRNA QC values were used to standardize the quantity of template used for QPCR amplification of target genes. Samples meeting quality control parameters were then used as the template for QPCR analysis of the target genes.

The Precision Profile™ for Osteoarthritis (Table 1) was selected through a synthesis of the literature on other OA gene expression studies and by review of Source MDx in-house datasets on OA and healthy patients (note that efforts to identify additional genes relevant to Osteoarthritis for inclusion in Table 1 are ongoing). Primer/probe sets were designed for the 30 genes listed in Table 1. All primer/probe reagents for the genes of interest were custom-designed in-house with the aid of Applied Biosystem's Primer Express® software to achieve three performance criteria: 1) single- gene specificity of amplification as tested by gel electrophoresis; 2) dilutional linearity of amplification performance over 5 orders of magnitude; and 3) amplification efficiency of 100+/−3% yielding a doubling of starting target material with each 1 CT unit decrease. Primer/probe sets were designed to span 90-120 base pairs with a preference toward the most 5′ forward design spanning an intron/exon junction. Primer designs were optimized for robust amplification, minimization of secondary hybridization, specificity and consistent performance. Quality-control testing of reagents and manufactured plates as described below helped to ensure that amplification specificity and efficiency remained within established metrics during storage and new synthesis of nucleotides.

Amplification specificity was tested by QPCR with a custom cDNA standard template of induced whole blood and cell lines, determining the size, number and DNA sequence of the amplified product. The size and number of amplified products was determined by agarose gel electrophoresis Amplified products were electrophoresed on a 4% agarose gel to visualize the number of DNA bands present. The molecular weight of each band was determined by comparison to known molecular weight markers (Fisher Scientific, no. PR-G1741, Hampton, N.H.). The presence of a single DNA band of the correct size was suggestive of specific amplification of the intended gene sequence. In certain cases, the amplified product DNA sequence was compared to the published sequence. Primer/probe amplification of genomic DNA was investigated using purified genomic DNA rather than cDNA as the template for QPCR. The formation of primer dimers and spurious amplification was also investigated using DEPC water as template for a “no template” control QPCR assay.

Amplification efficiency of a primer/probe set was determined by a dilutional linearity assay, using 5 serial dilutions of the standard cDNA template and running PCR reactions on each dilution in replicates of 4. Two versions of each target gene primer/probe set were designed and tested to select for both the amplification efficiency and specificity. Similarly, new primer/probe reagent lot performance was monitored to ensure matched amplification specificity and efficiency to previous primer/probe reagent lots. The primer/probe sets generate consistently repeatable results at less that 2% variation for control sets of cDNA.

Quantitative PCR was performed with the use of the ABI Prism 7900 Sequence Detector instruments. PCR reactions were run in 384-well plates and the intensity of the fluors measured. Each well also contained specific primers and probes to measure 18S rRNA, as an internal control. The amount of cDNA added to each reaction was held to a relatively narrow range, determined by the measurement of 18S RNA. Samples weremultiplexed, so that the CT for a constitutively expressed gene was used to calibrate the reaction. The difference CT(target)—CT(control) between the fluorescence threshold cycle (CT) for the target gene and the endogenous control (18S rRNA) is presented as a ACT value. For reference, a ACT of 2 is approximately equivalent to a 4-fold change in concentration of the transcript. The CT reporting system and estimation of relative gene expression is well described in the literature.

### Latent Class Modeling

Using Statistical Innovations consultants and software, models and algorithms were built to answer the following questions: 1) How do the OA subjects different at each time-point from the Normals, from themselves at other time-points, and from other OA patients at the gene-expression level? 2) Do the gene expressions predict clinical outcomes for OA subjects?

Logistic regression and latent class analyses was used to answer the above questions. An analysis began with determining significance of each gene using a logistic regression analysis. A latent class analysis builds discriminating models based on the ranking of a gene's significance. The latent class analysis discrimination determines group membership (i.e. normal vs. OA, progressor vs. non-progressor) as a function of the gene expression.

Traditional models used in regression, discriminant and log-linear analysis contain parameters that describe only relationships between observed variables. Latent class models, however, include discrete unobserved variables, such as change in expression at gene loci. Latent class models do not rely on traditional modeling assumptions, which are often violated in practice (linear relationship, normal distribution, homogeneity). Thus, they are less subject to biases associated with data not conforming to the assumptions of a model. Additionally, latent class models include variables of mixed scale types in the same analysis. This allows one to relate gene expression to the clinical indices and response to therapy (Magidson and Vermunt, 2005).

Briefly, to determine if OA subjects are different than normal, other diseases, and individually over-time within and between subjects, statistical differences at each gene loci (using ACT values at each loci) were determined. A ranking of the 30 genes from the Precision Profile for Osteoarthritis, from most to least significant is shown in Tables 4 and 5, which summarize the results of significance tests for the difference in the mean expression levels for normal subjects and subjects suffering from osteoarthritis. Since competing methods are available that are justified under different assumptions, p-values can be computed in 2 different ways.
1) Based on a 1-way ANOVA. This approach assumes that gene expression is normally distributed with the same variance within each population (Table 4).
2) Based on logistic regression, where group membership (Osteoarthritis v. Normal) is predicted as a function of the gene expression (Table 5). Conceptually, this is the reverse of what is done in the ANOVA approach where the gene expression is predicted as a function of the group. The logistic distribution holds true under several different distributional assumptions, including those that are made in the 1-way ANOVA approach. Therefore, the second strategy is justified under a more general class of distributional assumptions than the ANOVA approach.

As expected, the two different approaches yield comparable p-values and comparable rankings for the genes. As can be seen from Tables 4 and 5, the p-values are fairly similar for most genes except those having extremely low p-values, which include many of the low-expressing genes. For those, deviations from normal distributions may be responsible for the difference. The low-expressing genes (shaded gray in Tables 4 and 5) were excluded from the gene models. Strong predictive results were obtained without using the genes, as described below.

After excluding the under-expressing genes, the gene IL6R was found to be the most significant overall and was subject to further stepwise logistic regression analysis to generate 2-gene models capable of correctly classifying osteoarthritis and normal subjects with at least 75% accuracy, as described in Table 6 below.

Gene expression profiles were obtained using the 24 genes remaining after exclusion of the under-expressing genes using the SEARCH procedure in GOLDMineR, developed by Statistical Innovations (Magidson, 1998), to implement a stepwise logistic analysis for predicting the dichotomous variable that distinguishes subjects suffering from osteoarthritis from normal subjects, as a function of the 24 genes (unhighligted in Tables 3 and 4). The procedure enters the most significant gene into the logit model first, followed by the second, third and so on. The STEP analysis was performed under the assumption that the gene expressions follow a multinormal distribution, with different means and different variance-covariance matrices for the normal and osteoarthritis population.

Gene Expression Modeling using IL6R

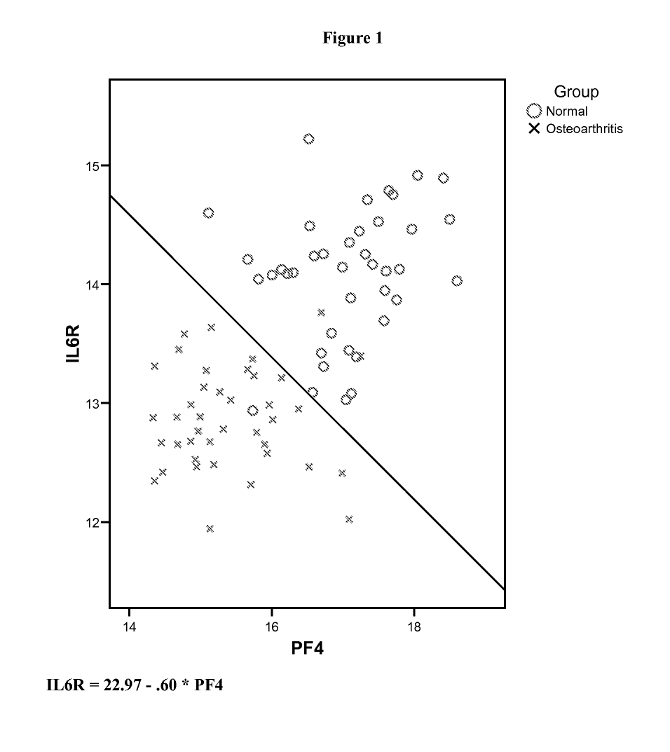

As previously described, IL6R was subject to further STEP analysis to identify multi-gene models capable of distinguishing between normal subjects versus subjects afflicted with osteoarthritis with at least 75% accuracy, where the 23 genes remaining (after exclusion of the under-expressing genes) were evaluated as the second gene in a 2-gene model. All models that yielded significant incremental p-values, at the 0.05 level, for the second gene were then analyzed using Latent Gold or Goldmine to find R² values. The R² statistic is a less formal statistical measure of goodness of prediction, which varies between 0 (predicted probability of having osteoarthritis is constant regardless of delta-ct values on the 2 genes) to 1 (predicted probability of having osteoarthritis =1 for each osteoarthritis subject, and =0 for each Normal subject). If the 2-gene model yielded an R² value greater than 0.6 it was kept as a model that discriminated well. If these models met the 0.6 cutoff, their statistical output from Latent Gold, was then used to determine classification percentages. As can be seen from Table 6, and FIG. 1, the 2-gene model IL6R and PF4 correctly classified subjects suffering from osteoarthritis or normal subjects with maximum classification rates of 95% and 98% accuracy, respectively. The ‘maximum overall rate’ is based on the predicted logit (predicted probability) cutoff that minimizes the total number of misclassifications in the sample.

The resulting 2-gene model, IL6R and PF4 is plotted in FIG. 1. FIG. 1 shows that a line can almost perfectly distinguish the two groups using the 2 gene model IL6R and PF4. This discrimination line is an example of the Index Function evaluated at a particular logit (log odds) value. Values above and to the right of the line are predicted to be in the normal, those below and to the left in the osteoarthritis population. This is a simplified version of the “Index function” as displayed in two dimensions.

The intercept (alpha) and slope (beta) of the discrimination line was computed according to the data shown in Table 7. A cutoff of 0.63 was used to compute alpha (equals 0.53222 in logit units).

The following equation describes the discrimination line shown in FIG. 1: Osteoarthritis Discrimination Line: IL6R=22.97−0.60*PF4.

Subjects below and to the left of this discrimination line have a predicted probability of being in the diseased group higher than the cutoff probability of 0.63.

The intercept C₀=22.97 was computed by taking the difference between the intercepts for the 2 groups [44.7661−(−44.7661)=89.5322] and subtracting the log-odds of the cutoff probability (0.53222). This quantity was then multiplied by −1/X where X is the coefficient for IL6R (-3.8746).

Gene Expression Modeling using IL6R

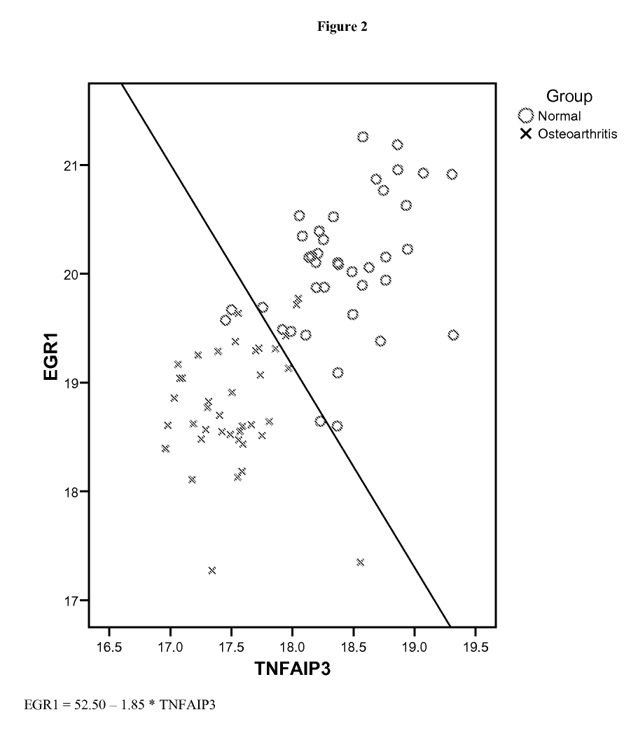

EGR1 was also subject to further STEP analysis to identify multi-gene models capable of distinguishing between normal subjects versus subjects afflicted with osteoarthritis with at least 75% accuracy, where the 23 genes remaining (after exclusion of the under-expressing genes) were evaluated as the second gene in a 2-gene model. As previously described, all models that yielded significant incremental p-values, at the 0.05 level, for the second gene were then analyzed using Latent Gold or Goldmine to find R² values. If the 2-gene model yielded an R² valuegreater than 0.6 it was kept as a model that discriminated well. If these models met the 0.6 cutoff, their statistical output from Latent Gold, was then used to determine classification percentages. As can be seen from Table 8, and FIG. 2, the 2-gene model EGR1 and TNFAIP3 correctly classified subjects suffering from osteoarthritis or normal subjects with maximum classification rates of 93% and 93% accuracy, respectively.

The resulting 2-gene model, EGR1 and TNFAIP3 is plotted in FIG. 2. FIG. 2 shows that a line can almost perfectly distinguish the two groups using the 2 gene model EGR1 and TNFAIP3. This discrimination line is an example of the Index Function evaluated at a particular logit (log odds) value. Values above and to the rightof the line are predicted to be in the normal, those below and to the left in the osteoarthritis population. This is a simplified version of the “Index function” as displayed in two dimensions.

The intercept (alpha) and slope (beta) of the discrimination line was computed according to the data shown in Table 9. A cutoff of 0.53 was used to compute alpha (equals 0.12014 in logit units).

The following equation describes the discrimination line shown in FIG. 2: Osteoarthritis Discrimination Line: EGR1=52.50−1.85*TNFAIP3.

Subjects below and to the left of this discrimination line have a predicted probability of being in the diseased group higher than the cutoff probability of 0.53.

The intercept C₀=52.5 was computed by taking the difference between the intercepts for the 2 groups [73.2065−(−73.2065)=146.413] and subtracting the log-odds of the cutoff probability (0.53222). This quantity was then multiplied by −1/X where X is the coefficient for EGR1 (−2.7866).

### Narrowing the Gene Panel Based on the Models

From the results obtained above, the number of genes in the OA gene panel is reduced and those genes identified in the models that discriminate OA from normal and predict clinical outcome are re-tested. These genes and models are tested/validated using an independent set of data from patients enrolled in the study (i.e. build models from data on the first 50 patients and test the model with data from the next set of 50 patients enrolled in the study).

These studies represent a first step in testing the ability of a gene expression panel to provide clinically-relevant information about OA disease activity and risk of progression. They are designed to test feasibility, initiate test validation and develop hypotheses. For these reasons, and an absence of any prior data in this field on which to predicate statistical power computations, no formal statistical justification of sample size have been undertaken. However, discussions with leaders in diagnostic development indicate that a sample size of 100 patients is generally necessary and appropriate to establish feasibility and primary validation.

Further analysis using the step-wise logit models can be used to predict clinical outcome within individual patients and populations of patients, examine differences in gene expression in non-responders vs. responders prior to dosing/change in medication, and examine gene expression prior to flare and change in disease activity status.

Additionally, models based on a larger panel of genes (-80), from samples obtained at the trial baseline, are developed. Samples from the first 50 patients are analyzed initially. The model is tested for generalizability using data from the remaining enrollees (N-50). Additionally, the model is tested using in-house gene expression data at Source MDx obtained from patients with inflammatory diseases such as lupus, MS, etc. This will determine if the model can discriminate between OA and inflammatory diseases. Further analysis is focused on models designed to monitor the OA patient using data collected at each time-point during the study.

The data described herein support that Gene Expression Profiles with sufficient precision and calibration as described herein (1) can determine subsets of individuals with a known biological condition, particularly individuals with osteoarthritis or individuals with conditions related to osteoarthritis; (2) may be used to monitor the response of patients to therapy; (3) may be used to assess the efficacy and safety of therapy; and (4) may be used to guide the medical management of a patient by adjusting therapy to bring one or more relevant Gene Expression Profiles closer to a target set of values, which may be normative values or other desired or achievable values.

Gene Expression Profiles are used for characterization and monitoring of treatment efficacy of individuals with osteoarthritis, or individuals with conditions related to osteoarthritis. Use of the algorithmic and statistical approaches discussed above to achieve such identification and to discriminate in such fashion is within the scope of various embodiments herein. The references listed below are hereby incorporated herein by reference.

### REFERENCES

Magidson, J. GOLDMineR User's Guide (1998). Belmont, Mass.: Statistical Innovations Inc.
Vermunt J. K. and J. Magidson. Latent GOLD 4.0 User's Guide. (2005) Belmont, Mass.: Statistical Innovations Inc.
Vermunt J. K. and J. Magidson. Technical Guide for Latent GOLD 4.0: Basic and Advanced (2005)
Belmont, Mass.: Statistical Innovations Inc.
Vermunt J. K. and J. Magidson. Latent Class Cluster Analysis in (2002) J. A. Hagenaars and A. L. McCutcheon (eds.), Applied Latent Class Analysis, 89-106. Cambridge: Cambridge University Press.
Magidson, J. “Maximum Likelihood Assessment of Clinical Trials Based on an Ordered Categorical Response.” (1996) Drug Information Journal, Maple Glen, Pa.: Drug Information Association, Vol. 30, No. 1, pp 143-170.

TABLE 1
Precision Profile ™ for Osteoarthritis:

| Gene | | Gene Accession |
|---|---|---|
| Symbol | Gene Name | Number |
| EGR1 | early growth response 1 | NM_001964 |
| IFNG | interferon gamma | NM_000619 |
| IFNGR1 | interferon gamma receptor 1 | NM_000416 |
| IL10 | interleukin 10 | NM_000572 |
| IL12B | interleukin 12B (natural killer cell | NM_002187 |
| | stimulatory factor 2, cytotoxic | |
| | lymphocyte maturation factor 2, p40) | |
| IL13 | Interleukin 13 | NM_002188 |
| IL13RA1 | interleukin 13 receptor, alpha | NM_001560 |
| IL18 | Interleukin 18 | NM_001562 |
| IL18BP | IL-18 Binding Protein | NM_005699 |
| IL18R1 | interleukin 18 receptor 1 | NM_003855 |
| IL1A | interleukin 1, alpha | NM_000575 |
| IL1B | Interleukin 1, beta | NM_000576 |
| IL1R1 | interleukin 1 receptor, type I | NM_000877 |
| IL1RN | interleukin 1 receptor antagonist | NM_173843 |
| IL2 | Interleukin 2 | NM_000586 |
| IL4 | interleukin 4 | NM_000589 |
| IL4R | interleukin 4 receptor | NM_000418 |
| IL6 | interleukin 6 (interferon, beta 2) | NM_000600 |
| IL6R | interleukin 6 receptor | NM_000565 |
| IL8 | interleukin 8 | NM_000584 |
| MMP9 | matrix metallopeptidase 9 (gelatinase B, | NM_004994 |
| | 92 kDa gelatinase, 92 kDa | |
| | type IV collagenase) | |
| PF4 | platelet factor 4 (chemokine | NM_002619 |
| | (C—X—C motif) ligand 4) | |
| TGFB1 | transforming growth factor, beta 1 | NM_000660 |
| | (Camurati-Engelmann disease) | |
| TGFB3 | Transforming growth factor, beta 3 | NM_003239 |
| TGFBR1 | transforming growth factor, beta | NM_004612 |
| | receptor I (activin A receptor | |
| | type II-like kinase, 53 kDa) | |
| TGFBR2 | Tranforming growth factor, | NM_003242 |
| | beta receptor II | |
| TNF | tumor necrosis factor | NM_000594 |
| | (TNF superfamily, member 2) | |
| TNFAIP3 | tumor necrosis factor, alpha-induced | NM_006290 |
| | protein 3 | |
| TNFAIP6 | tumor necrosis factor, alpha-induced | NM_007115 |
| | protein 6 | |
| TNFRSF1A | tumor necrosis factor receptor | NM_001065 |
| | superfamily, member 1A | |

TABLE 2
Precision Profile ™ for Inflammatory Response

| Gene | | Gene Accession |
|---|---|---|
| Symbol | Gene Name | Number |
| ADAM17 | a disintegrin and metalloproteinase domain 17 (tumor | NM_003183 |
| | necrosis factor, alpha, converting enzyme) | |
| ALOX5 | arachidonate 5-lipoxygenase | NM_000698 |
| ANXA11 | annexin A11 | NM_001157 |
| APAF1 | apoptotic Protease Activating Factor 1 | NM_013229 |
| BAX | BCL2-associated X protein | NM_138761 |
| C1QA | complement component 1, q subcomponent, alpha | NM_015991 |
| | polypeptide | |
| CASP1 | caspase 1, apoptosis-related cysteine peptidase (interleukin | NM_033292 |
| | 1, beta, convertase) | |
| CASP3 | caspase 3, apoptosis-related cysteine peptidase | NM_004346 |
| CCL2 | chemokine (C-C motif) ligand 2 | NM_002982 |
| CCL3 | chemokine (C-C motif) ligand 3 | NM_002983 |
| CCL5 | chemokine (C-C motif) ligand 5 | NM_002985 |
| CCR3 | chemokine (C-C motif) receptor 3 | NM_001837 |
| CCR5 | chemokine (C-C motif) receptor 5 | NM_000579 |
| CD14 | CD14 antigen | NM_000591 |
| CD19 | CD19 Antigen | NM_001770 |
| CD4 | CD4 antigen (p55) | NM_000616 |
| CD86 | CD86 antigen (CD28 antigen ligand 2, B7-2 antigen) | NM_006889 |
| CD8A | CD8 antigen, alpha polypeptide | NM_001768 |
| CRP | C-reactive protein, pentraxin-related | NM_000567 |
| CSF2 | colony stimulating factor 2 (granulocyte-macrophage) | NM_000758 |
| CSF3 | colony stimulating factor 3 (granulocytes) | NM_000759 |
| CTLA4 | cytotoxic T-lymphocyte-associated protein 4 | NM_005214 |
| CXCL1 | chemokine (C—X—C motif) ligand 1 (melanoma growth | NM_001511 |
| | stimulating activity, alpha) | |
| CXCL10 | chemokine (C—X—C moif) ligand 10 | NM_001565 |
| CXCL3 | chemokine (C—X—C motif) ligand 3 | NM_002090 |
| CXCL5 | chemokine (C—X—C motif) ligand 5 | NM_002994 |
| CXCR3 | chemokine (C—X—C motif) receptor 3 | NM_001504 |
| DPP4 | Dipeptidylpeptidase 4 | NM_001935 |
| EGR1 | early growth response-1 | NM_001964 |
| ELA2 | elastase 2, neutrophil | NM_001972 |
| FAIM3 | Fas apoptotic inhibitory molecule 3 | NM_005449 |
| FASLG | Fas ligand (TNF superfamily, member 6) | NM_000639 |
| GCLC | glutamate-cysteine ligase, catalytic subunit | NM_001498 |
| GZMB | granzyme B (granzyme 2, cytotoxic T-lymphocyte-associated | NM_004131 |
| | serine esterase 1) | |
| HLA-DRA | major histocompatibility complex, class II, DR alpha | NM_019111 |
| HMGB1 | high-mobility group box 1 | NM_002128 |
| HMOX1 | heme oxygenase (decycling) 1 | NM_002133 |
| HSPA1A | heat shock protein 70 | NM_005345 |
| ICAM1 | Intercellular adhesion molecule 1 | NM_000201 |
| ICOS | inducible T-cell co-stimulator | NM_012092 |
| IFI16 | interferon inducible protein 16, gamma | NM_005531 |
| IFNG | interferon gamma | NM_000619 |
| IL10 | interleukin 10 | NM_000572 |
| IL12B | interleukin 12 p40 | NM_002187 |
| IL13 | interleukin 13 | NM_002188 |
| IL15 | Interleukin 15 | NM_000585 |
| IRF1 | interferon regulatory factor 1 | NM_002198 |
| IL18 | interleukin 18 | NM_001562 |
| IL18BP | IL-18 Binding Protein | NM_005699 |
| IL1A | interleukin 1, alpha | NM_000575 |
| IL1B | interleukin 1, beta | NM_000576 |
| IL1R1 | interleukin 1 receptor, type I | NM_000877 |
| IL1RN | interleukin 1 receptor antagonist | NM_173843 |
| IL2 | interleukin 2 | NM_000586 |
| IL23A | interleukin 23, alpha subunit p19 | NM_016584 |
| IL32 | interleukin 32 | NM_001012631 |
| IL4 | interleukin 4 | NM_000589 |
| IL5 | interleukin 5 (colony-stimulating factor, eosinophil) | NM_000879 |
| IL6 | interleukin 6 (interferon, beta 2) | NM_000600 |
| IL8 | interleukin 8 | NM_000584 |
| LTA | lymphotoxin alpha (TNF superfamily, member 1) | NM_000595 |
| MAP3K1 | mitogen-activated protein kinase kinase kinase 1 | XM_042066 |
| MAPK14 | mitogen-activated protein kinase 14 | NM_001315 |
| MHC2TA | class II, major histocompatibility complex, transactivator | NM_000246 |
| MIF | macrophage migration inhibitory factor (glycosylation- | NM_002415 |
| | inhibiting factor) | |
| MMP12 | matrix metallopeptidase 12 (macrophage elastase) | NM_002426 |
| MMP8 | matrix metallopeptidase 8 (neutrophil collagenase) | NM_002424 |
| MMP9 | matrix metallopeptidase 9 (gelatinase B, 92 kDa gelatinase, | NM_004994 |
| | 92 kDa type IV collagenase) | |
| MNDA | myeloid cell nuclear differentiation antigen | NM_002432 |
| MPO | myeloperoxidase | NM_000250 |
| MYC | v-myc myelocytomatosis viral oncogene homolog (avian) | NM_002467 |
| NFKB1 | nuclear factor of kappa light polypeptide gene enhancer in B- | NM_003998 |
| | cells 1 (p105) | |
| NOS2A | nitric oxide synthase 2A (inducible, hepatocytes) | NM_000625 |
| PLA2G2A | phospholipase A2, group IIA (platelets, synovial fluid) | NM_000300 |
| PLA2G7 | phospholipase A2, group VII (platelet-activating factor | NM_005084 |
| | acetylhydrolase, plasma) | |
| PLAU | plasminogen activator, urokinase | NM_002658 |
| PLAUR | plasminogen activator, urokinase receptor | NM_002659 |
| PRTN3 | proteinase 3 (serine proteinase, neutrophil, Wegener | NM_002777 |
| | granulomatosis autoantigen) | |
| PTGS2 | prostaglandin-endoperoxide synthase 2 (prostaglandin G/H | NM_000963 |
| | synthase and cyclooxygenase) | |
| PTPRC | protein tyrosine phosphatase, receptor type, C | NM_002838 |
| PTX3 | pentraxin-related gene, rapidly induced by IL-1 beta | NM_002852 |
| SERPINA1 | serine (or cysteine) proteinase inhibitor, clade A (alpha-1 | NM_000295 |
| | antiproteinase, antitrypsin), member 1 | |
| SERPINE1 | serpin peptidase inhibitor, clade E (nexin, plasminogen | NM_000602 |
| | activator inhibitor type 1), member 1 | |
| SSI-3 | suppressor of cytokine signaling 3 | NM_003955 |
| TGFB1 | transforming growth factor, beta 1 (Camurati-Engelmann | NM_000660 |
| | disease) | |
| TIMP1 | tissue inhibitor of metalloproteinase 1 | NM_003254 |
| TLR2 | toll-like receptor 2 | NM_003264 |
| TLR4 | toll-like receptor 4 | NM_003266 |
| TNF | tumor necrosis factor (TNF superfamily, member 2) | NM_000594 |
| TNFRSF13B | tumor necrosis factor receptor superfamily, member 13B | NM_012452 |
| TNFRSF17 | tumor necrosis factor receptor superfamily, member 17 | NM_001192 |
| TNFRSF1A | tumor necrosis factor receptor superfamily, member 1A | NM_001065 |
| TNFSF13B | Tumor necrosis factor (ligand) superfamily, member 13b | NM_006573 |
| TNFSF5 | CD40 ligand (TNF superfamily, member 5, hyper-IgM | NM_000074 |
| | syndrome) | |
| TXNRD1 | thioredoxin reductase | NM_003330 |
| VEGF | vascular endothelial growth factor | NM_003376 |

TABLE 3
Vitamin D/Knee OA Trial: Schedule Of Visits And Examinations

| Visit Time (months) | −1 | 0 | 2 | 4 | 8 | 12 | 16 | 20 | 24 | P.T.† |
|---|---|---|---|---|---|---|---|---|---|---|
| Visit Type/Number | Screen | Baseline | f/u | f/u | f/u | f/u | f/u | f/u | f/u | P.T.† |
| Consents | X | | | | | | | | | |
| History & Physical | X | | | | | | | | | |
| General Physical Exam | | | | | | X | | | X | X |
| Eligibility | X | X | | | | | | | | |
| Randomization | | X | | | | | | | | |
| Pill dispensation | X^{¶} | X | | X | X | X | X | X | | |
| Pill return | | X | | X | X | X | X | X | X | X |
| Diary/Calendar/Journal | | X | | X | X | X | X | X | X | X |
| review | | | | | | | | | | |
| Adverse Event | | | | X | X | X | X | X | X | X |
| Questionnaire | | | | | | | | | | |
| Knee Exam | X | X | | | | X | | | X | X |
| CBC, ESR | X | | | | | | | | | |
| Serum Biochemical Panel | X | | | | | | | | | |
| Serum total calcium and | X | X | | X | X | X | X | X | X | |
| albumin | | | | | | | | | | |
| Spot urine Ca: creatinine | | X | X | X | X | X | X | X | X | |
| ratio (safely)# | | | | | | | | | | |
| Serum 25(OH)D level | X | X | | X | X | X | | | X | X |
| Serum PTH level | | X | | | | X | | | X | X |
| Knee X-Ray: PA semi- | X | | | | | | | | X | X |
| flexed | | | | | | | | | | |
| Knee MRI | | X | | | | X | | | X | X |
| WOMAC | X pain | X | | X | X | X | X | X | X | X |
| SF-36 | | X | | | | X | | | X | X |
| Physical Functional Tests | | X | | | | X | | | X | X |
| Blood Collection for | | X | X | (X¹) | X | X | X | X | X | X |
| PAXGENE Analysis (2.5 mls) | | | | | | | | | | |

TABLE 4
Ranking of genes based on Table 1 from most to
least significant: 1-Way ANOVA Approach

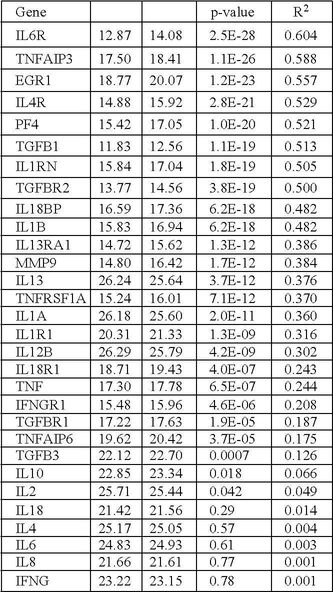

TABLE 5
Ranking of genes based on Table 1 from most to
least significant: Stepwise logistic regression

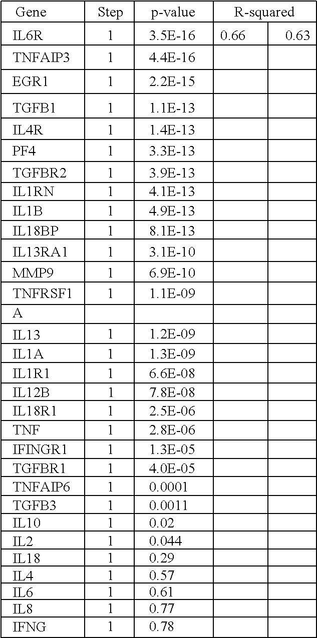

TABLE 6
2-gene Models based on genes from Table 1 using IL6R
as the initial gene 2 Gene

| | % Osteoarthritisr | % Normal |
|---|---|---|
| Maximum = | 95% | 98% |

| | | GM | GM | LG |
|---|---|---|---|---|
| | STEP | P-Value | R² | R² |
| IL6R | 1 | 3.5E−16 | 0.66 | 0.63 |
| PF4 | 2 | 3.6E−05 | 0.80 | 0.81 |

TABLE 7
Discrimination Line for IL6R and PF4
Osteoarthritis vs Normals—IL6R PF4

| | | | | |
|---|---|---|---|---|
| R² | 0.8138 | | | |
| Group | Class1 | | | |
| Intercept | | | 0.63 = | cutoff |
| Normal | −44.7661 | | 0.53222 = | logit(cutoff) |
| Osteoarthritis | 44.7661 | | | |
| | | alpha = | 22.9701 | |
| Predictors | Class1 | | 10.9916 | |
| IL6R | −3.8746 | | | |
| PF4 | −2.3206 | beta = | −0.59893 | |

TABLE 8
2-gene Models based on genes from Table 1 using EGR1
as the initial gene
2 Gene

| | % Osteoarthritis | % Normal |
|---|---|---|
| Maximum = | 93% | 93% |

| | | GM | GM | LG |
|---|---|---|---|---|
| | STEP | P-Value | R² | R² |
| EGR1 | 1 | 2.2E−15 | 0.63 | 0.64 |
| TNFAIP3 | 2 | 1.5E−05 | 0.78 | 0.76 |

TABLE 9
Discrimination Line for EGR1 and TNFAIP3
Osteoarthritis vs Normals—EGR1
TNFAIP3

| | | | | |
|---|---|---|---|---|
| R² | 0.7586 | | | |
| Group | Class1 | | | |
| Intercept | | | 0.53 = | cutoff |
| Normal | −73.2065 | | 0.12014 = | logit(cutoff) |
| Osteoarthritis | 73.2065 | | | |
| | | alpha = | 52.4987 | |
| Predictors | Class1 | | 15.4485 | |
| EGR1 | −2.7866 | | | |
| TNFAIP3 | −5.1622 | beta = | −1.85251 | |

What is claimed is:

1. A method of evaluting the presence of osteoarthritis or a condition related to osteoarthritis in a subject, based on a sample from the subject, the sample providing a source of RNAs, the method comprising:
a) using amplification for determining a quantitative measure of the amount of at least two constituents as distinct RNA constituents in the subject sample, wherein the first constituent is TNFAIP3 or IL6R, and the second constituent is selected from the group consisting of: IL6R, TNFAIP3, EGR1, TGFB1, IL4R, PF4, TGFBR2, IL1RN, IL1B, IL18BP, IL13RA1, MMP9, TNFRSF1A, IL1R1, IL18R1, TNF, IFNGR1, TGFBR1, TNFAIP6, TGFB3, and IL10, and wherein such measure is obtained under measurement conditions that are substantially repeatable and the constituents are selected so that measurement of the constituents distinguishes between a normal subject and an osteoarthritis-diagnosed subject in a reference population with at least 75% accuracy; and
b) comparing the quantitative measure of the constituents in the subject sample to a reference value.

2. A method of evaluting the presence of osteoarthritis or a condition related to osteoarthritis in a subject, based on a sample from the subject, the sample providing a source of RNAs, the method comprising:
a) using amplification for determining a quantitative measure of the amount of at least two constituents of any constituent of Table 1 or Table 2 as distinct RNA constituents in the subject sample, wherein such measure is obtained under measurement conditions that are substantially repeatable and the constituents are selected so that measurement of the constituents distinguishes between a normal subject and an osteoarthritis-diagnosed subject in a reference population with at least 75% accuracy; and
b) comparing the quantitative measure of the constituents in the subject sample to a reference value.

3. A method for determining a profile data set for characterizing a subject with osteoarthritis or a condition related to osteoarthritis, based on a sample from the subject, the sample providing a source of RNAs, the method comprising:
a) using amplification for measuring the amount of RNA in a panel of constituents including at least two constituents from Table 1 or Table 2 as distinct RNA constituents in the subject sample and
b) arriving at a measure of each constituent,
wherein the profile data set comprises the measure of each constituent of the panel and wherein amplification is performed under measurement conditions that are substantially repeatable.

4. A method for assessing or monitoring the response to therapy in a subject having osteoarthritis based on a sample from the subject, the sample providing a source of RNAs, comprising:
a) determining a quantitative measure of the amount of at least two constituents of any constituent of Table 1 or Table 2 as distinct RNA constituents, wherein such measure is obtained under measurement conditions that are substantially repeatable to produce subject data set; and
b) comparing the subject data set to a baseline data set.

5. A method for monitoring the progression of osteoarthritis in a subject, based on a sample from the subject, the sample providing a source of RNAs, comprising:
a) determining a quantitative measure of the amount of at least two constituents of any constituent of Table 1 or Table 2 as distinct RNA constituents in a sample obtained at a first period of time, wherein such measure is obtained under measurement conditions that are substantially repeatable to produce a first subject data set;
b) determining a quantitative measure of the amount of at least two constituents of any constituent of Table 1 or Table 2 as distinct RNA constituents in a sample obtained at a second period of time, wherein such measure is obtained under measurement conditions that are substantially repeatable to produce a second subject data set; and
c) comparing the first subject data set and the second subject data set.

6. The method of any of claims 2, wherein the at least two constituents are selected from the group consisting of: IL6R, TNFAIP3, EGR1, TGFB1, IL4R, PF4, TGFBR2, IL1RN, IL1B, IL18BP, IL13RA1, MMP9, TNFRSF1A, IL1R1, IL18R1, TNF, IFNGR1, TGFBR1, TNFAIP6, TGFB3, and IL10.

7. The method of claim 6, comprising determining a quantitative measure of at least IL6R.

8. The method of claim 7, further comprising determining a quantitative measure of PF4.

9. The method of claim 6, comprising determining a quantitative measure of at least EGR1.

10. The method of claim 9, further comprising determining a quantitative measure of TNFAIP3.

11. The method of any of claims 1, wherein expression of said constituents in said subject is increased compared to expression of said constituents in a normal reference sample.

12. The method of any of claims 1, wherein expression of said constituents in said subject is decreased compared to expression of said constituents in a normal reference sample.

13. The method of claim 4, wherein when the baseline data set is derived from
a) a normal subject, a similarity in the subject data set and the baseline date set indicates that said therapy is efficacious; or
b) a subject known to have osteoarthritis, a similarity in the subject data set and the baseline date set indicates that said therapy is not efficacious.

14. The method of any of claims 1-5, wherein said subject sample is selected from the group consisting of blood, a blood fraction, a body fluid, a cell and a tissue.

15. The method according to any of claims 1, wherein the measurement conditions that are substantially repeatable are within a degree of repeatability of better than five percent.

16. The method of claim 15, wherein the measurement conditions that are substantially repeatable are within a degree of repeatability of better than three percent.

17. The method of any of claims 1 wherein efficiencies of amplification for all constituents are substantially similar.

18. The method of any of claims 1, wherein the efficiency of amplification for all constituents is within ten percent or less.

19. The method of claim 18, wherein the efficiency of amplification for all constituents is within five percent or less.

20. The method of claim 19, wherein the efficiency of amplification for all constituents is within three percent or less.

21. A kit for detecting osteoarthritis in a subject, comprising at least one reagent for the detection or quantification of any constituent measured according to claim 1 and instructions for using the kit.
